(19) United States
(12) Patent Application Publication
(10) Pub. No.: US 2026/0284156 A1
(43) Pub. Date: Sep. 24, 2026

(54) Compositions and Methods for Treating Celiac Sprue Disease

(71) Applicant: University of Washington Through Its Center For Commercializaiton, Seattle, WA (US)

(72) Inventors: Justin SIEGEL, Seattle, WA (US); David BAKER, Seattle, WA (US); Sydney Rin Anna GORDON, Seattle, WA (US); Ingrid Swanson PULTZ, Seattle, WA (US); Elizabeth Joy STANLEY, Bothell, WA (US); Sarah Jane WOLF, Camas, WA (US)

(21) Appl. No.: 19/444,902

(22) Filed: Jan. 9, 2026

Related U.S. Application Data
(63) Continuation of application No. 18/513,879, filed on Nov. 20, 2023, now Pat. No. 12,544,430.
Application No. 18/513,879 is a continuation of application No. 17/114,294, filed on Dec. 7, 2020, now abandoned.
(63) Continuation of application No. 16/203,109, filed on Nov. 28, 2018, now Pat. No. 10,874,720.
Application No. 16/203,109 is a continuation of application No. 15/896,536, filed on Feb. 14, 2018, now Pat. No. 10,149,892.
Application No. 15/896,536 is a continuation of application No. 15/633,065, filed on Jun. 26, 2017, now Pat. No. 9,925,249.
Application No. 15/633,065 is a continuation of application No. 15/006,341, filed on Jan. 26, 2016, now Pat. No. 9,707,280.
Application No. 15/006,341 is a division of application No. 14/131,601, filed on Aug. 10, 2012, now Pat. No. 9,289,473.
(60) Provisional application No. 61/521,899, filed on Aug. 10, 2011.

(51) Int. Cl. A61K 38/48; C12N 9/52

(52) U.S. Cl. CPC A61K 38/482; A61K 38/48; C12N 9/52; C12Y 304/21

(57) ABSTRACT

The invention provides compositions and methods for treating celiac sprue.

### CROSS REFERENCE

This application is a Continuation of U.S. patent application Ser. No. 18/513,879, filed Nov. 20, 2023 which is a Continuation of U.S. patent application Ser. No. 17/114,294, filed Dec. 7, 2020 now abandoned, which is a Continuation of U.S. patent application Ser. No. 16/203,109, filed Nov. 28, 2018 now U.S. patent Ser. No. 10/874,720 issued Dec. 29, 2020, which is a Continuation of U.S. patent application Ser. No. 15/896,536, filed Feb. 14, 2018 now U.S. patent Ser. No. 10/149,892 issued Dec. 11, 2018, which is a Continuation of U.S. patent application Ser. No. 15/633,065, filed Jun. 26, 2017 now U.S. Pat. No. 9,925,249 issued Mar. 27, 2018, which is a Continuation of U.S. patent application Ser. No. 15/006,341, filed Jan. 26, 2016 now U.S. Pat. No. 9,707,280 issued Jul. 18, 2017, which is a Divisional of U.S. patent application Ser. No. 14/131,601, filed Feb. 26, 2014 now U.S. Pat. No. 9,289,473 issued Mar. 22, 2016, which an International Application No. PCT/US2012/050364, filed Aug. 10, 2012, which claims priority to U.S. Provisional Application No. 61/521,899, filed Aug. 10, 2011, the disclosures of which are hereby incorporated by reference in their entirety.

### STATEMENT OF GOVERNMENT SUPPORT

This invention was made with government support under Defense Advanced Research Projects Agency (DARPA) grant number HR0011-08-1-0085. The government has certain rights in the invention.

### SEQUENCE LISTING STATEMENT

A computer readable form of the Sequence Listing is filed with this application by electronic submission and is incorporated into this application by reference in its entirety. The Sequence Listing is contained in the file created on Nov. 15, 2023, having the file name “12-709-WO-US-DIV5.xml” and is 151,972 bytes in size.

### BACKGROUND

Celiac sprue is a highly prevalent disease in which dietary proteins found in wheat, barley, and rye products known as ‘glutens’ evoke an immune response in the small intestine of genetically predisposed individuals. The resulting inflammation can lead to the degradation of the villi of the small intestine, impeding the absorption of nutrients. Symptoms can appear in early childhood or later in life, and range widely in severity, from diarrhea, fatigue and weight loss to abdominal distension, anemia, and neurological symptoms. There are currently no effective therapies for this lifelong disease except the total elimination of glutens from the diet. Although celiac sprue remains largely underdiagnosed, its' prevalence in the US and Europe is estimated at 0.5-1.0% of the population. The identification of suitable naturally-occurring enzymes as oral therapeutics for Celiac disease is difficult due to the stringent physical and chemical requirements to specifically and efficiently degrade gluten-derived peptides in the harsh and highly acidic environment of the human digestive tract.

### SUMMARY OF THE INVENTION

In a first aspect, the present invention provides polypeptides comprising an amino acid sequence at least 75% identical to an amino acid sequence according to SEQ ID NO:35, wherein
(a) the polypeptide degrades a PQPQLP (SEQ ID NO:34) peptide at pH 4;
(b) residue 278 is Ser, residue 78 is Glu, and residue 82 is Asp; and
(c) the polypeptide comprises an amino acid change from SEQ ID NO: 67 at one or more residues selected from the group consisting of 73, 102, 103, 104, 130, 165, 168, 169, 172, and 179.

In a second aspect, the present invention provides polypeptide comprising an amino acid sequence at least 75% identical to an amino acid sequence according to SEQ ID NO:1, wherein
(a) the polypeptide degrades a PQPQLP (SEQ ID NO:34) peptide at pH 4;
(b) residue 467 is Ser, residue 267 is Glu, and residue 271 is Asp; and
(c) the polypeptide comprises an amino acid change from SEQ ID NO: 33 at one or more residues selected from the group consisting of 119, 262, 291, 292, 293, 319, 354, 357, 358, 361, and 368.

In various embodiments of the first and second aspect, the polypeptide comprises an amino acid sequence at least 85%, 95%, or 100% identical to an amino acid sequence according to SEQ ID NO:1 or SEQ ID NO: 35. In another embodiment, the polypeptide, comprises an amino acid sequence according to any one of SEQ ID NO:2-66.

In another aspect, the present invention provides polypeptides comprising an amino acid sequence according to SEQ ID NO:1, wherein the polypeptide comprises at least one amino acid change from SEQ ID NO: 33. In another aspect, the present invention provides a polypeptide comprising an amino acid sequence according to SEQ ID NO:35, wherein the polypeptide comprises at least one amino acid change from SEQ ID NO: 67. In various embodiments, the polypeptide, comprises an amino acid sequence according to any one of SEQ ID NO:2-66.

In a further aspect, the present invention provides nucleic acids encoding the polypeptide of any aspect or embodiment of the invention. In another aspect, the invention provides nucleic acid expression vectors comprising the isolated nucleic acids of the invention. In a further embodiment, the invention provides recombinant host cells comprising the nucleic acid expression vectors of the invention. In another aspect, the invention provides pharmaceutical compositions, comprising the polypeptides, the nucleic acids, the nucleic acid expression vectors and/or the recombinant host cells of the invention, and a pharmaceutically acceptable carrier.

In another aspect, the invention provides methods for treating celiac sprue, comprising administering to an individual with celiac sprue a polypeptide or pharmaceutical composition according to any embodiment of the invention, or a polypeptide comprising an amino acid selected from the group consisting of SEQ ID NO:33 or SEQ ID NO:67.

### BRIEF DESCRIPTION OF THE FIGURES

FIG. 1. Schematic depicting the role of enzyme therapeutics in the treatment of Celiac disease. Gluten is comprised of many glycoproteins including α-gliadin. Partial proteolysis of α-gliadin (SEQ ID NO: 71) results in protease-resistant peptides enriched in a PQ dipeptide motif that can lead to inflammation and disease. The addition of an oral enzyme therapeutic that is functional in the stomach and capable of specifically degrading the immunogenic peptides could potentially act as a therapeutic for this disease.

FIG. 2. Computational models of the peptide binding sites for KumaWT and KumaMax. A) KumaWT in complex with a PR dipeptide motif. B) KumaMax in complex with the designed PQ dipeptide motif. Computationally designed residues in the active site are labeled and highlighted in sticks. The modeled peptides were based on a bound form of Kumamolisin-AS (PDB ID: 1T1E) and final structures were generated using the Rosetta Molecular Modeling Suite. Images were generated using PyMol v1.5.

FIG. 3. Protein stability after incubation with pepsin or trypsin. Stability was measured by quantifying the relative remaining fraction of intact protein as observed on an SDS-PAGE gel after 30 minutes of incubation in the presence or absence of pepsin or trypsin at the pH indicated. Each protein was measured in triplicate and the error bars represent the standard deviation. Quantification was performed in ImageJ™.

FIG. 4. An immunogenic α9-gliadin peptide is degraded by KumaMax. A) Reaction chromatograms measuring the abundance of the M+H ion of the parent 09-gliadin peptide after 50 minutes of incubation with no enzyme, SC PEP, or KumaMax™. B) The fraction of α9-gliadin peptide remaining in the presence of KumaMax™ as a function of incubation time at pH 4. The data was fit using a standard exponential decay function. The R² value was greater than 0.9.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2^{nd} Ed. (R. I. Freshney. 1987. Liss, Inc. New York, NY), Gene Transfer and Expression Protocols, pp. 109-128, ed. E. J. Murray, The Humana Press Inc., Clifton, N.J.), and the Ambion 1998 Catalog (Ambion, Austin, TX).

As used herein, the singular forms “a”, “an” and “the” include plural referents unless the context clearly dictates otherwise. “And” as used herein is interchangeably used with “or” unless expressly stated otherwise.

As used herein, amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the invention can be used in combination, unless the context clearly dictates otherwise.

In a first aspect, the present invention provides polypeptides comprising an amino acid sequence at least 75% identical to an amino acid sequence according to SEQ ID NO:35, wherein
(a) the polypeptide degrades a PQPQLP (SEQ ID NO:34) peptide at pH 4;
(b) residue 278 is Ser, residue 78 is Glu, and residue 82 is Asp; and
(c) the polypeptide comprises an amino acid change from SEQ ID NO: 67 at one or more residues selected from the group consisting of 73, 102, 103, 104, 130, 165, 168, 169, 172, and 179.

As disclosed in the examples that follow, polypeptides according to this aspect of the invention can be used, for example, in treating celiac sprue. The polypeptides are modified versions of either the processed version of Kumamolisin-As (SEQ ID NO:67) or the preprocessed version of Kumamolisin-As (SEQ ID NO:33), which is known as a member of the sedolisin family of serine-carboxyl peptidases, and utilizes the key catalytic triad Ser²⁷⁸-Glu⁷⁸-Asp⁸² in its processed form to hydrolyze its substrate (Ser⁴⁶⁷-Gu²⁶⁷-Asp²⁷¹ in the pre-processed form) Its maximal activity is at pH ~4.0. While the native substrate for Kumamolisin-As is unknown, it has been previously shown to degrade collagen under acidic conditions (4). In addition, this enzyme has been shown to be thermostable, with an ideal temperature at 60° C., but still showing significant activity at 37° C.

The inventors of the present invention have unexpectedly discovered that Kumamolisin-As is capable of degrading proline (P)- and glutamine (Q)-rich components of gluten known as ‘gliadins’ believed responsible for the bulk of the immune response in most celiac sprue patients. The polypeptides of the invention show improved protease activity at pH 4 against the oligopeptide PQPQLP (a substrate representative of gliadin) compared to wild type Kumamolisin-As.

The polypeptides of this aspect of the invention degrade a PQPQLP (SEQ ID NO:34) peptide at pH 4. Such degradation occurs under the conditions disclosed in the examples that follow.

The polypeptides of this aspect comprise one or more amino acid changes from SEQ ID NO: 67 (wild type processed Kumamolisin-As) at one or more residues selected from the group consisting of residues 73, 102, 103, 104, 130, 165, 168, 169, 172, and 179 (numbering based on the wild type processed Kumamolisin-As amino acid sequence). In non-limiting embodiments, the one or more changes relative to the wild type processed Kumamolisin-As amino acid sequence (SEQ ID NO:67) are selected from the group consisting of:

| WT Residue# | AA change |
|---|---|
| S73 | K, G |
| N102 | D |
| T103 | S |
| D104 | A, T, N |
| G130 | S |
| S165 | N |
| T168 | A |
| D169 | N, G |
| Q172 | D |
| D179 | S, H |

In various further non-limiting embodiments, the one or more changes relative to the wild type processed Kumamolisin-As amino acid sequence include at least N102D. In another embodiment the one or more changes relative to the wild type Kumamolisin-As amino acid sequence include at least N102D and D169N or D169G. In another embodiment the one or more changes relative to the wild type Kumamolisin-As amino acid sequence include at least N102D, D169G, and D179H. In another embodiment the one or more changes relative to the wild type Kumamolisin-As amino acid sequence include at least S73K, D104T, N102D, G130S, D169G, and D179H.

As used herein, “at least 75% identical” means that the polypeptide differs in its full length amino acid sequence by less 25% or less (including any amino acid substitutions, deletions, additions, or insertions) from the polypeptide defined by SEQ ID NO:35.

In various preferred embodiment, the polypeptide s comprise or consist of an amino acid sequence at least 76%, 77%, 78%, 79%, 80%, 81%, 82%, 83%, 84%, 85%, 86%, 87%, 88%, 89%, 90%, 91%, 92%, 92%, 93%, 94%, 95%, 96%, 97%, 98%, or 99% identical to an amino acid sequence according to SEQ ID NO:35. In a further embodiment the polypeptides comprise or consist of an amino acid sequence according to SEQ ID NO:35.

In various further embodiments, the polypeptides comprise or consist of an amino acid sequence at least 75% identical to any one of SEQ ID NOS:36-66. The polypeptides represented by these SEQ ID NOS are specific examples of polypeptides with improved protease activity at pH 4 against the oligopeptide PQPQLP (SEQ ID NO: 34) (a substrate representative of gliadin) compared to wild type Kumamolisin-As. In various preferred embodiment, the polypeptide s comprise or consist of an amino acid sequence at least 76%, 77%, 78%, 79%, 80%, 81%, 82%, 83%, 84%, 85%, 86%, 87%, 88%, 89%, 90%, 91%, 92%, 92%, 93%, 94%, 95%, 96%, 97%, 98%, or 99% identical to an amino acid sequence according to any one of SEQ ID NOS:36-66. In a further embodiment the polypeptides comprise or consist of an amino acid sequence according to any one of SEQ ID NOS:36-66.

In a preferred embodiment of this first aspect, the polypeptides comprising an amino acid sequence at least 75% identical to an amino acid sequence according to SEQ ID NO:1 (based on variants of the preprocessed version of Kumamolisin-As), wherein
(a) the polypeptide degrades a PQPQLP (SEQ ID NO:34) peptide at pH 4;
(b) residue 467 is Ser, residue 267 is Glu, and residue 271 is Asp; and
(c) the polypeptide comprises an amino acid change from SEQ ID NO: 33 at one or more residues selected from the group consisting of 119, 262, 291, 292, 293, 319, 354, 357, 358, 361, and 368.

The polypeptides of this embodiment comprise one or more amino acid changes from SEQ ID NO: 33 (wild type pre-processed Kumamolisin-As) at one or more residues selected from the group consisting of residues 119, 262, 291, 292, 293, 319, 354, 357, 358, 361, and 368 (numbering based on the wild type pre-processed Kumamolisin-As amino acid sequence). In non-limiting embodiments, the one or more changes relative to the wild type Kumamolisin-As amino acid sequence are selected from the group consisting of:

| WT Residue# | AA change |
|---|---|
| V119 | D |
| S262 | K, G |
| N291 | D |
| T292 | S |
| D293 | A, T, N |
| G319 | S |
| S354 | N |
| T357 | A |
| D358 | N, G |
| Q361 | D |
| D368 | S, H |

In various further non-limiting embodiments, the one or more changes relative to the wild type Kumamolisin-As amino acid sequence include at least N291D. In another embodiment the one or more changes relative to the wild type Kumamolisin-As amino acid sequence include at least N291D and 358N or 358G. In another embodiment the one or more changes relative to the wild type Kumamolisin-As amino acid sequence include at least N291D, 358G, and 368H. In another embodiment the one or more changes relative to the wild type Kumamolisin-As amino acid sequence include at least V119D, S262K, D293T, N291D, G319S, D358G, and D368H.

As used herein, “at least 75% identical” means that the polypeptide differs in its full length amino acid sequence by less 25% or less (including any amino acid substitutions, deletions, additions, or insertions) from the polypeptide defined by SEQ ID NO:1.

In various preferred embodiment, the polypeptide s comprise or consist of an amino acid sequence at least 76%, 77%, 78%, 79%, 80%, 81%, 82%, 83%, 84%, 85%, 86%, 87%, 88%, 89%, 90%, 91%, 92%, 92%, 93%, 94%, 95%, 96%, 97%, 98%, or 99% identical to an amino acid sequence according to SEQ ID NO:1. In a further embodiment the polypeptides comprise or consist of an amino acid sequence according to SEQ ID NO:1.

In various further embodiments, the polypeptides comprise or consist of an amino acid sequence at least 75% identical to any one of SEQ ID NOS:2-32. The polypeptides represented by these SEQ ID NOS are specific examples of polypeptides with improved protease activity at pH 4 against the oligopeptide PQPQLP (SEQ ID NO: 34) (a substrate representative of gliadin) compared to wild type Kumamolisin-As. In various preferred embodiment, the polypeptide s comprise or consist of an amino acid sequence at least 76%, 77%, 78%, 79%, 80%, 81%, 82%, 83%, 84%, 85%, 86%, 87%, 88%, 89%, 90%, 91%, 92%, 92%, 93%, 94%, 95%, 96%, 97%, 98%, or 99% identical to an amino acid sequence according to any one of SEQ ID NOS:2-32. In a further embodiment the polypeptides comprise or consist of an amino acid sequence according to any one of SEQ ID NOS:2-32.

In a second aspect, the present invention provides polypeptides comprising or consisting of an amino acid sequence according to SEQ ID NO:35, wherein the polypeptide comprises at least one amino acid change from SEQ ID NO: 67. As disclosed in the examples that follow, polypeptides according to this aspect of the invention can be used, for example, in treating celiac sprue. The polypeptides are modified versions of processed Kumamolisin-As (SEQ ID NO:67), that show improved protease activity at pH 4 against the oligopeptide PQPQLP (SEQ ID NO: 34) (a substrate representative of gliadin) compared to wild type Kumamolisin-As. In one embodiment, the polypeptides comprise or consist of an amino acid sequence according to SEQ ID NO:36. Polypeptides according to SEQ ID NO:36 have a N102D mutation relative to wild-type processed Kumamolisin-As. As shown in the examples that follow, polypeptides containing this mutation have at least 10-fold improved protease activity at pH 4 against the oligopeptide PQPQLP (SEQ ID NO: 34) compared to wild type Kumamolisin-As.

In another embodiment of this second aspect, the polypeptides comprise or consist of an amino acid sequence according to SEQ ID NO:37. Polypeptides according to SEQ ID NO:37 have a N102D mutation and a D169N or D169G mutation relative to wild-type processed Kumamolisin-As. As shown in the examples that follow, polypeptides containing this mutation have at least 20-fold improved protease activity at pH 4 against the oligopeptide PQPQLP (SEQ ID NO: 34) compared to wild type Kumamolisin-As.

In another embodiment of this second aspect, the polypeptides comprise or consist of an amino acid sequence according to SEQ ID NO:38. Polypeptides according to SEQ ID NO:38 have a N102D mutation, a D169G, and a D179H mutation relative to wild-type processed Kumamolisin-As. As shown in the examples that follow, polypeptides containing this mutation have at least 50-fold improved protease activity at pH 4 against the oligopeptide PQPQLP (SEQ ID NO: 34) compared to wild type Kumamolisin-As.

In a further embodiment of this second aspect, the polypeptides comprise or consist of an amino acid sequence according to any one of SEQ ID NOS:39-66. Polypeptides according to these embodiments have all been demonstrated to show improved protease activity at pH 4 against the oligopeptide PQPQLP (SEQ ID NO: 34) compared to wild type Kumamolisin-As. In a preferred embodiment, the polypeptide comprises or consists of an amino acid sequence according to SEQ ID NO:66.

In a preferred embodiment of this second aspect, the present invention provides polypeptides comprising or consisting of an amino acid sequence according to SEQ ID NO:1, wherein the polypeptide comprises at least one amino acid change from SEQ ID NO: 33. As disclosed in the examples that follow, polypeptides according to this aspect of the invention can be used, for example, in treating celiac sprue. The polypeptides are modified versions of preprocessed Kumamolisin-As (SEQ ID NO:33), that show improved protease activity at pH 4 against the oligopeptide PQPQLP (SEQ ID NO: 34) (a substrate representative of gliadin) compared to wild type Kumamolisin-As. In one embodiment, the polypeptides comprise or consist of an amino acid sequence according to SEQ ID NO:2. Polypeptides according to SEQ ID NO:2 have a N291D mutation relative to preprocessed wild-type Kumamolisin-As. As shown in the examples that follow, polypeptides containing this mutation have at least 10-fold improved protease activity at pH 4 against the oligopeptide PQPQLP (SEQ ID NO: 34) compared to wild type Kumamolisin-As.

In another embodiment of this second aspect, the polypeptides comprise or consist of an amino acid sequence according to SEQ ID NO:3. Polypeptides according to SEQ ID NO:3 have a N291D mutation and a D358N or D358G mutation relative to preprocessed wild-type Kumamolisin-As. As shown in the examples that follow, polypeptides containing this mutation have at least 20-fold improved protease activity at pH 4 against the oligopeptide PQPQLP (SEQ ID NO: 34) compared to wild type Kumamolisin-As.

In another embodiment of this second aspect, the polypeptides comprise or consist of an amino acid sequence according to SEQ ID NO:4. Polypeptides according to SEQ ID NO:4 have a N291D mutation, a D358G, and a D368H mutation relative to preprocessed wild-type Kumamolisin-As. As shown in the examples that follow, polypeptides containing this mutation have at least 50-fold improved protease activity at pH 4 against the oligopeptide PQPQLP (SEQ ID NO: 34) compared to wild type Kumamolisin-As.

In a further embodiment of this second aspect, the polypeptides comprise or consist of an amino acid sequence according to any one of SEQ ID NOS:5-32. Polypeptides according to these embodiments have all been demonstrated to show improved protease activity at pH 4 against the oligopeptide PQPQLP (SEQ ID NO: 34) compared to wild type Kumamolisin-As. In a preferred embodiment, the polypeptide comprises or consists of an amino acid sequence according to SEQ ID NO:32; this polypeptide is shown in the examples that follow to possess the most potent protease activity at pH 4 against the oligopeptide PQPQLP (SEQ ID NO: 34) of any of the polypeptides tested.

As used throughout the present application, the term “polypeptide” is used in its broadest sense to refer to a sequence of subunit amino acids, whether naturally occurring or of synthetic origin. The polypeptides of the invention may comprise L-amino acids, D-amino acids (which are resistant to L-amino acid-specific proteases in vivo), or a combination of D- and L-amino acids. The polypeptides described herein may be chemically synthesized or recombinantly expressed. The polypeptides may be linked to other compounds to promote an increased half-life in vivo, such as by PEGylation, HESylation, PASylation, or glycosylation. Such linkage can be covalent or non-covalent as is understood by those of skill in the art. The polypeptides may be linked to any other suitable linkers, including but not limited to any linkers that can be used for purification or detection (such as FLAG or His tags).

In a third aspect, the present invention provides isolated nucleic acids encoding the polypeptide of any aspect or embodiment of the invention. The isolated nucleic acid sequence may comprise RNA or DNA. As used herein, “isolated nucleic acids” are those that have been removed from their normal surrounding nucleic acid sequences in the genome or in cDNA sequences. Such isolated nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the invention.

In a fourth aspect, the present invention provides nucleic acid expression vectors comprising the isolated nucleic acid of any embodiment of the invention operatively linked to a suitable control sequence. “Recombinant expression vector” includes vectors that operatively link a nucleic acid coding region or gene to any control sequences capable of effecting expression of the gene product. “Control sequences” operably linked to the nucleic acid sequences of the invention are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type known in the art, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The construction of expression vectors for use in transfecting prokaryotic cells is also well known in the art, and thus can be accomplished via standard techniques. (See, for example, Sambrook, Fritsch, and Maniatis, in: Molecular Cloning, A Laboratory Manual, Cold Spring Harbor Laboratory Press, 1989; Gene Transfer and Expression Protocols, pp. 109-128, ed. E. J. Murray, The Humana Press Inc., Clifton, N.J.), and the Ambion 1998 Catalog (Ambion, Austin, TX). The expression vector must be replicable in the host organisms either as an episome or by integration into host chromosomal DNA. In a preferred embodiment, the expression vector comprises a plasmid. However, the invention is intended to include other expression vectors that serve equivalent functions, such as viral vectors.

In a fifth aspect, the present invention provides recombinant host cells comprising the nucleic acid expression vectors of the invention. The host cells can be either prokaryotic or eukaryotic. The cells can be transiently or stably transfected or transduced. Such transfection and transduction of expression vectors into prokaryotic and eukaryotic cells can be accomplished via any technique known in the art, including but not limited to standard bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press; Culture of Animal Cells: A Manual of Basic Technique, 2^{nd} Ed. (R. I. Freshney. 1987. Liss, Inc. New York, NY). A method of producing a polypeptide according to the invention is an additional part of the invention. The method comprises the steps of (a) culturing a host according to this aspect of the invention under conditions conducive to the expression of the polypeptide, and (b) optionally, recovering the expressed polypeptide. The expressed polypeptide can be recovered from the cell free extract, cell pellet, or recovered from the culture medium. Methods to purify recombinantly expressed polypeptides are well known to the man skilled in the art.

In a sixth aspect, the present invention provides pharmaceutical compositions, comprising the polypeptide, nucleic acid, nucleic acid expression vector, and/or the recombinant host cell of any aspect or embodiment of the invention, and a pharmaceutically acceptable carrier. The pharmaceutical compositions of the invention can be used, for example, in the methods of the invention described below. The pharmaceutical composition may comprise in addition to the polypeptides, nucleic acids, etc. of the invention (a) a lyoprotectant; (b) a surfactant; (c) a bulking agent; (d) a tonicity adjusting agent; (e) a stabilizer; (f) a preservative and/or (g) a buffer.

In some embodiments, the buffer in the pharmaceutical composition is a Tris buffer, a histidine buffer, a phosphate buffer, a citrate buffer or an acetate buffer. The pharmaceutical composition may also include a lyoprotectant, e.g. sucrose, sorbitol or trehalose. In certain embodiments, the pharmaceutical composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof in other embodiments, the pharmaceutical composition includes a bulking agent, like glycine. In yet other embodiments, the pharmaceutical composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The pharmaceutical composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the pharmaceutical composition additionally includes a stabilizer, e.g., a molecule which, when combined with a protein of interest substantially prevents or reduces chemical and/or physical instability of the protein of interest in lyophilized or liquid form. Exemplary stabilizers include sucrose, sorbitol, glycine, inositol, sodium chloride, methionine, arginine, and arginine hydrochloride.

The polypeptides, nucleic acids, etc. of the invention may be the sole active agent in the pharmaceutical composition, or the composition may further comprise one or more other active agents suitable for an intended use.

The pharmaceutical compositions described herein generally comprise a combination of a compound described herein and a pharmaceutically acceptable carrier, diluent, or excipient. Such compositions are substantially free of non-pharmaceutically acceptable components, i.e., contain amounts of non-pharmaceutically acceptable components lower than permitted by US regulatory requirements at the time of filing this application. In some embodiments of this aspect, if the compound is dissolved or suspended in water, the composition further optionally comprises an additional pharmaceutically acceptable carrier, diluent, or excipient. In other embodiments, the pharmaceutical compositions described herein are solid pharmaceutical compositions (e.g., tablet, capsules, etc.).

These compositions can be prepared in a manner well known in the pharmaceutical art, and can be administered by any suitable route. In a preferred embodiment, the pharmaceutical compositions and formulations are designed for oral administration. Conventional pharmaceutical carriers, aqueous, powder or oily bases, thickeners and the like may be necessary or desirable.

The pharmaceutical compositions can be in any suitable form, including but not limited to tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

In a seventh aspect, the present invention provides methods for treating celiac sprue, comprising administering to an individual with celiac sprue an amount effective to treat the celiac sprue of one or more polypeptides selected from the group consisting of the polypeptides of the first or second aspects of the invention, SEQ ID NO:33, and SEQ ID NO:67.

The inventors of the present invention have unexpectedly discovered that Kumamolisin-As is capable of degrading proline (P)- and glutamine (Q)-rich components of gluten known as ‘gliadins’ believed responsible for the bulk of the immune response in most celiac sprue patients. The polypeptides of the invention show improved protease activity at pH 4 against the oligopeptide PQPQLP (SEQ ID NO: 34) (a substrate representative of gliadin) compared to wild type Kumamolisin-As.

In one embodiment, the one or more polypeptides comprise an amino acid sequence at least 75% identical to an amino acid sequence according to SEQ ID NO:35, wherein
(a) the polypeptide degrades a PQPQLP (SEQ ID NO:34) peptide at pH 4;
(b) residue 278 is Ser, residue 78 is Glu, and residue 82 is Asp.

In further embodiments, the one or more polypeptides comprise one or more amino acid changes from SEQ ID NO: 67 (wild type processed Kumamolisin-As) at one or more residues selected from the group consisting of residues 73, 102, 103, 104, 130, 165, 168, 169, 172, and 179 (numbering based on the wild type processed Kumamolisin-As amino acid sequence). In non-limiting embodiments, the one or more changes relative to the wild type processed Kumamolisin-As amino acid sequence (SEQ ID NO:67) are selected from the group consisting of:

| WT Residue# | AA change |
|---|---|
| S73 | K, G |
| N102 | D |
| T103 | S |
| D104 | A, T, N |
| G130 | S |
| S165 | N |
| T168 | A |
| D169 | N, G |
| Q172 | D |
| D179 | S, H |

In various further non-limiting embodiments, the one or more changes relative to the wild type processed Kumamolisin-As amino acid sequence include at least N102D. In another embodiment the one or more changes relative to the wild type Kumamolisin-As amino acid sequence include at least N102D and D169N or D169G. In another embodiment the one or more changes relative to the wild type Kumamolisin-As amino acid sequence include at least N102D, D169G, and D179H. In another embodiment the one or more changes relative to the wild type Kumamolisin-As amino acid sequence include at least S73K, D104T, N102D, G130S, D169G, and D179H.

In various preferred embodiment, the one or more polypeptide s comprise or consist of an amino acid sequence at least 76%, 77%, 78%, 79%, 80%, 81%, 82%, 83%, 84%, 85%, 86%, 87%, 88%, 89%, 90%, 91%, 92%, 92%, 93%, 94%, 95%, 96%, 97%, 98%, or 99% identical to an amino acid sequence according to SEQ ID NO:35. In a further embodiment the polypeptides comprise or consist of an amino acid sequence according to SEQ ID NO:35.

In various further embodiments, the one or more polypeptides comprise or consist of an amino acid sequence at least 75% identical to any one of SEQ ID NOS:36-66. The polypeptides represented by these SEQ ID NOS are specific examples of polypeptides with improved protease activity at pH 4 against the oligopeptide PQPQLP (SEQ ID NO: 34) (a substrate representative of gliadin) compared to wild type Kumamolisin-As. In various preferred embodiment, the polypeptide s comprise or consist of an amino acid sequence at least 76%, 77%, 78%, 79%, 80%, 81%, 82%, 83%, 84%, 85%, 86%, 87%, 88%, 89%, 90%, 91%, 92%, 92%, 93%, 94%, 95%, 96%, 97%, 98%, or 99% identical to an amino acid sequence according to any one of SEQ ID NOS:36-66. In a further embodiment the polypeptides comprise or consist of an amino acid sequence according to any one of SEQ ID NOS:36-66.

In a preferred embodiment, the polypeptides for use in the methods of this aspect of the invention comprise an amino acid according to SEQ ID NO:33 or a polypeptide comprising one or more amino acid changes from SEQ ID NO: 33 (wild type preprocessed Kumamolisin-As) at one or more residues selected from the group consisting of residues 119, 262, 291, 292, 293, 319, 354, 357, 358, 361, and 368 (numbering based on the wild type Kumamolisin-As amino acid sequence). In non-limiting embodiments, the one or more changes relative to the wild type Kumamolisin-As amino acid sequence are selected from the group consisting of:

| WT Residue# | AA change |
|---|---|
| V119 | D |
| S262 | K, G |
| N291 | D |
| T292 | S |
| D293 | A, T, N |
| G319 | S |
| S354 | N |
| T357 | A |
| D358 | N, G |
| Q361 | D |
| D368 | S, H |

In various further non-limiting preferred embodiments, the one or more changes relative to the wild type Kumamolisin-As amino acid sequence include at least N291D. In another embodiment the one or more changes relative to the wild type Kumamolisin-As amino acid sequence include at least N291D and 358N or 358G. In another embodiment the one or more changes relative to the wild type Kumamolisin-As amino acid sequence include at least N291D, 358G, and 368H. In another embodiment the one or more changes relative to the wild type Kumamolisin-As amino acid sequence include at least V119D, S262K, D293T, N291D, G319S, D358G, and D368H.

In various preferred embodiment, the polypeptide s for use in the methods of this aspect of the invention comprise or consist of an amino acid sequence at least 76%, 77%, 78%, 79%, 80%, 81%, 82%, 83%, 84%, 85%, 86%, 87%, 88%, 89%, 90%, 91%, 92%, 92%, 93%, 94%, 95%, 96%, 97%, 98%, or 99% identical to an amino acid sequence according to SEQ ID NO:1. In a further embodiment the polypeptides comprise or consist of an amino acid sequence according to SEQ ID NO:1.

In various further embodiments, the polypeptides for use in the methods of this aspect of the invention comprise or consist of an amino acid sequence at least 75% identical to any one of SEQ ID NOS:2-32. The polypeptides represented by these SEQ ID NOS are specific examples of polypeptides with improved protease activity at pH 4 against the oligopeptide PQPQLP (SEQ ID NO: 34) (a substrate representative of gliadin) compared to wild type Kumamolisin-As. In various preferred embodiment, the polypeptides for use in the methods of this aspect of the invention comprise or consist of an amino acid sequence at least 76%, 77%, 78%, 79%, 80%, 81%, 82%, 83%, 84%, 85%, 86%, 87%, 88%, 89%, 90%, 91%, 92%, 92%, 93%, 94%, 95%, 96%, 97%, 98%, or 99% identical to an amino acid sequence according to any one of SEQ ID NOS:2-32. In a further embodiment the polypeptides comprise or consist of an amino acid sequence according to any one of SEQ ID NOS:2-32.

In an eighth aspect, the present invention provides methods for treating celiac sprue, comprising administering to an individual with celiac sprue a polypeptide comprising an amount effective of amino acid sequence according to any one of SEQ ID NOS: 1-67 to treat the celiac sprue.

In one embodiment, the polypeptides administered comprise or consist of an amino acid sequence according to SEQ ID NO:2. Polypeptides according to SEQ ID NO:2 have a N291D mutation relative to wild-type Kumamolisin-As. As shown in the examples that follow, polypeptides containing this mutation have at least 10-fold improved protease activity at pH 4 against the oligopeptide PQPQLP (SEQ ID NO: 34) compared to wild type Kumamolisin-As.

In another embodiment of this second aspect, the polypeptides administered comprise or consist of an amino acid sequence according to SEQ ID NO:3. Polypeptides according to SEQ ID NO:3 have a N291D mutation and a D358N or D358G mutation relative to wild-type Kumamolisin-As. As shown in the examples that follow, polypeptides containing this mutation have at least 20-fold improved protease activity at pH 4 against the oligopeptide PQPQLP (SEQ ID NO: 34) compared to wild type Kumamolisin-As.

In another embodiment of this second aspect, the polypeptides administered comprise or consist of an amino acid sequence according to SEQ ID NO:4. Polypeptides according to SEQ ID NO:4 have a N291D mutation, a D358G, and a D368H mutation relative to wild-type Kumamolisin-As. As shown in the examples that follow, polypeptides containing this mutation have at least 50-fold improved protease activity at pH 4 against the oligopeptide PQPQLP (SEQ ID NO: 34) compared to wild type Kumamolisin-As.

In a further embodiment of this second aspect, the polypeptides administered comprise or consist of an amino acid sequence according to any one of SEQ ID NOS:5-32. Polypeptides according to these embodiments have all been demonstrated to show improved protease activity at pH 4 against the oligopeptide PQPQLP (SEQ ID NO: 34) compared to wild type Kumamolisin-As. In a preferred embodiment, the polypeptide administered comprises or consists of an amino acid sequence according to SEQ ID NO:32; this polypeptide is shown in the examples that follow to possess the most potent protease activity at pH 4 against the oligopeptide PQPQLP (SEQ ID NO: 34) of any of the polypeptides tested.

In another embodiment, the one or more polypeptides comprise an amino acid sequence according to SEQ ID NO:36. Polypeptides according to SEQ ID NO:36 have a N102D mutation relative to wild-type processed Kumamolisin-As. As shown in the examples that follow, polypeptides containing this mutation have at least 10-fold improved protease activity at pH 4 against the oligopeptide PQPQLP (SEQ ID NO: 34) compared to wild type Kumamolisin-As. In another embodiment, the one or more polypeptides comprise an amino acid sequence according to SEQ ID NO:37. Polypeptides according to SEQ ID NO:37 have a N102D mutation and a D169N or D169G mutation relative to wild-type processed Kumamolisin-As. As shown in the examples that follow, polypeptides containing this mutation have at least 20-fold improved protease activity at pH 4 against the oligopeptide PQPQLP (SEQ ID NO: 34) compared to wild type Kumamolisin-As. In another embodiment, the one or more polypeptides comprise an amino acid sequence according to SEQ ID NO:38. Polypeptides according to SEQ ID NO:38 have a N102D mutation, a D169G, and a D179H mutation relative to wild-type processed Kumamolisin-As. As shown in the examples that follow, polypeptides containing this mutation have at least 50-fold improved protease activity at pH 4 against the oligopeptide PQPQLP (SEQ ID NO: 34) compared to wild type Kumamolisin-As. In further embodiments, the one or more polypeptides comprise an amino acid sequence according to any one of SEQ ID NOS:39-66. Polypeptides according to these embodiments have all been demonstrated to show improved protease activity at pH 4 against the oligopeptide PQPQLP (SEQ ID NO: 34) compared to wild type Kumamolisin-As. In a preferred embodiment, the polypeptide comprises or consists of an amino acid sequence according to SEQ ID NO:66.

Celiac sprue (also known as celiac disease or gluten intolerance) is a highly prevalent disease in which dietary proteins found in wheat, barley, and rye products known as ‘glutens’ evoke an immune response in the small intestine of genetically predisposed individuals. The resulting inflammation can lead to the degradation of the villi of the small intestine, impeding the absorption of nutrients. Symptoms can appear in early childhood or later in life, and range widely in severity, from diarrhea, fatigue, weight loss, abdominal pain, bloating, excessive gas, indigestion, constipation, abdominal distension, nausea/vomiting, anemia, bruising easily, depression, anxiety, growth delay in children, hair loss, dermatitis, missed menstrual periods, mouth ulcers, muscle cramps, joint pain, nosebleeds, seizures, tingling or numbness in hands or feet, delayed puberty, defects in tooth enamel, and neurological symptoms such as ataxia or paresthesia. There are currently no effective therapies for this lifelong disease except the total elimination of glutens from the diet. Although celiac sprue remains largely underdiagnosed, its' prevalence in the US and Europe is estimated at 0.5-1.0% of the population.

As used herein, “treating celiac sprue” means accomplishing one or more of the following: (a) reducing the severity of celiac sprue; (b) limiting or preventing development of symptoms characteristic of celiac sprue; (c) inhibiting worsening of symptoms characteristic of celiac sprue; (d) limiting or preventing recurrence of celiac sprue in patients that have previously had the disorder; (e) limiting or preventing recurrence of symptoms in patients that were previously symptomatic for celiac sprue; and (f) limiting development of celiac sprue in a subject at risk of developing celiac sprue, or not yet showing the clinical effects of celiac sprue.

The individual to be treated according to the methods of the invention may be any individual suffering from celiac sprue, including human subjects. The individual may be one already suffering from symptoms or one who is asymptomatic.

As used herein, an “amount effective” refers to an amount of the polypeptide that is effective for treating celiac sprue. The polypeptides are typically formulated as a pharmaceutical composition, such as those disclosed above, and can be administered via any suitable route, including orally, parentally, by inhalation spray, or topically in dosage unit formulations containing conventional pharmaceutically acceptable carriers, adjuvants, and vehicles. In a preferred embodiment, the pharmaceutical compositions and formulations are orally administered, such as by tablets, pills, lozenges, elixirs, suspensions, emulsions, solutions, or syrups.

Dosage regimens can be adjusted to provide the optimum desired response (e.g., a therapeutic or prophylactic response). A suitable dosage range may, for instance, be 0.1 ug/kg-100 mg/kg body weight; alternatively, it may be 0.5 ug/kg to 50 mg/kg; 1 ug/kg to 25 mg/kg, or 5 ug/kg to 10 mg/kg body weight. The polypeptides can be delivered in a single bolus, or may be administered more than once (e.g., 2, 3, 4, 5, or more times) as determined by an attending physician.

### Examples

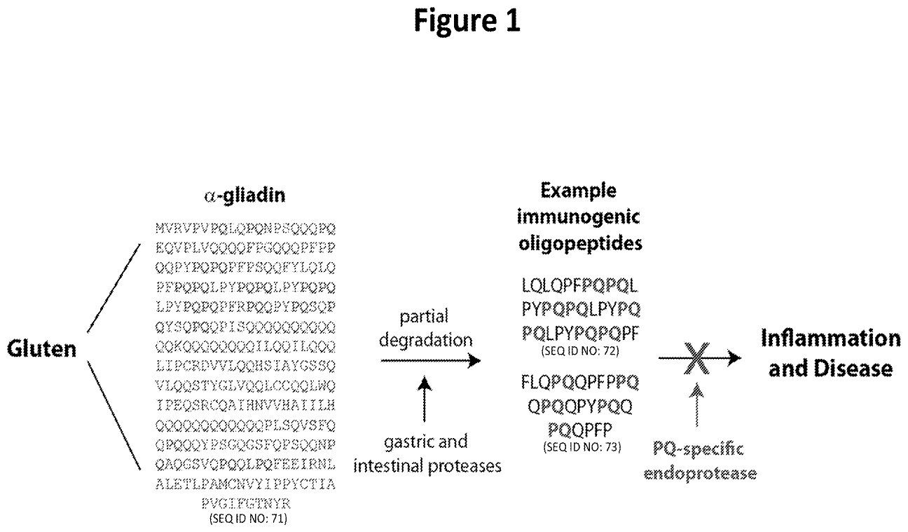

Celiac disease is an autoimmune disorder that afflicts approximately 1% of the population^{1,2}. This disease is characterized by an inflammatory reaction to gluten, the major protein in wheat flour, and to related proteins in barley and rye². Gluten is composed of a heterogeneous mixture of the glycoproteins gliadin and glutenin-. Upon ingestion, α-gliadin is partially degraded by gastric and intestinal proteases to oligopeptides, which are resistant to further proteolysis due to their unusually high proline and glutamine content³. (FIG. 1). Immunogenic oligopeptides that result from incomplete proteolysis are enriched in the PQ motif^{4,5} (FIG. 1), which stimulate inflammation and injury in the intestine of people with Celiac disease. Currently the only treatment for this disease is complete elimination of gluten from the diet, which is difficult to attain due to the ubiquity of this protein in modern food products⁶.

Oral enzyme therapy (OET) in which orally administered proteases are employed to hydrolyze immunogenic peptides before they are capable of triggering inflammation is currently being explored as a treatment for gluten intolerance. For this purpose, several different proteases have been considered due to their specificity for cleavage after either proline or glutamine residues^{4,7-9}. However, these enzymes often demonstrate characteristics that hinder their use in OET for gluten degradation. Most of these peptidases exhibit optimal catalytic activity at neutral pH; however, the pH of the human stomach ranges from 2 to 4. These enzymes are therefore most active when they reach the pH-neutral small intestine, which is too late for effective prevention of Celiac disease as this is the site where gluten-derived pathology develops^{2,10}. Additionally, several of these enzymes demonstrate instability in the low pH of the human stomach, are susceptible to proteolysis by digestive proteases, or require extensive refolding procedures during their purification⁷⁻¹¹, which are all characteristics that hamper efforts for clinical use.

The ideal protease for the application of OET in the treatment of gluten intolerance would combine the following traits: optimal activity at low pH, easy purification, stability under the conditions of the human stomach, and high specificity for amino acid motifs found in gluten-derived immunogenic oligopeptides. Here we report the engineering of an endopeptidase that demonstrates these traits. We identified a protease that is highly active in acidic conditions, Kumamolisin-As (KumaWT) from the acidophilic bacterium Alicyclobacillus sendaiensis, and used computational modeling tools to engineer it toward the desired oligopeptide specificity. The computationally designed enzyme, designated KumaMax™, exhibited over 100-fold increased proteolytic activity and an 800-fold switch in substrate specificity for the targeted PQ motif compared to wild-type KumaWT. In addition, KumaMax™ demonstrates resistance to common gastric proteases and is produced at high yields in E. coli without the need for refolding. Thus, this protease and others reported herein represent promising therapeutic candidates for Celiac disease.

### Results

### Selection and Computational Design of an α-Gliadin Endopeptidase

In order to engineer a novel protease that can degrade gluten peptides under gastric conditions, we first focused on identifying an appropriate protease as a starting point for our engineering efforts. Ideally, the template protease would combine stability and activity at low pH with demonstrated specificity for a dipeptide amino acid motif We identified the enzyme Kumamolisin-As (KumaWT) as a template, since this protease naturally has an optimal activity over the pH range of 2-4¹², which matches the approximate pH ranges in the human stomach before and after a meal is ingested (pH 2 and 4, respectively)¹³. KumaWT also demonstrates high stability and activity at the physiologically relevant temperature 37° C.¹⁴. In addition, the purification of this enzyme is straightforward and yields significant quantities using standard recombinant protein production methods in E. coli¹⁴, an important property both for screening mutant libraries and for its ultimate generation in large batches for use in OET. Finally, KumaWT naturally recognizes a specific dipeptide motif as opposed to single amino acid specificity¹⁴. This is an important property for an oral protease therapeutic meant to be taken during digestion, since dipeptide specificity should result in reduced competitive inhibition by other food-derived peptides in the stomach.

An effective OET for Celiac disease would likely demonstrate specificity for Proline-Glutamine (PQ), due to the frequent occurrence of this dipeptide in immunogenic gluten-derived oligopeptides (FIG. 1). KumaWT has a strong specificity for proline at the P2 position of its peptide substrate, matching one of the amino acid residues of interest for the degradation of immunogenic α-gliadin peptides. In the P1 site, KumaWT has been established to prefer the positively charged amino acids arginine or lysine¹⁴. Despite this preference, KumaWT is also capable of recognizing glutamine at the P1 position, albeit at a significantly decreased level compared to its recognition of arginine or lysine¹⁴. This slight innate proclivity to recognize glutamine at the P1 position suggests that KumaWT may be amenable to re-engineering to prefer glutamine at this position. At the P1′ site, KumaWT demonstrates broad specificity, which is desirable since the residue in the position after the PQ motif varies among the different immunogenic peptides, as depicted in FIG. 1.

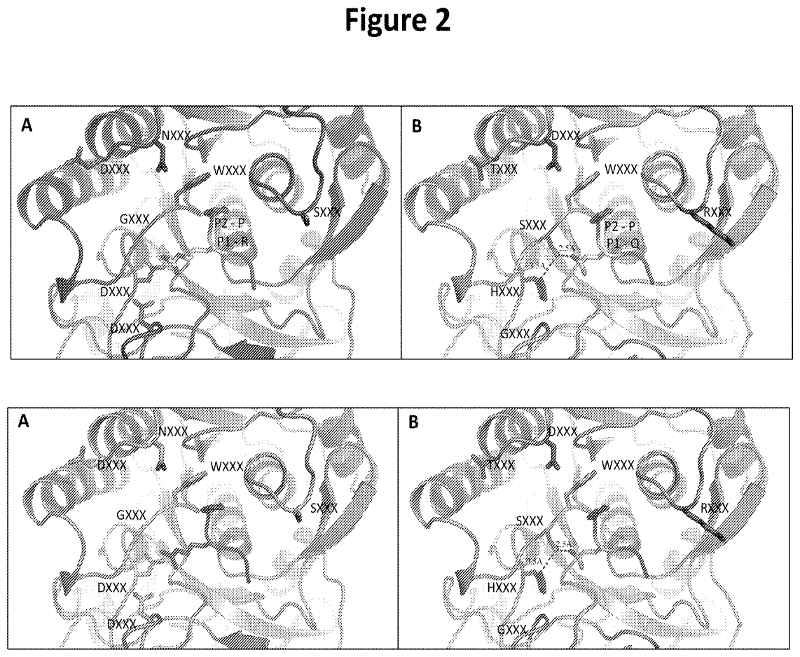

Given these characteristics of KumaWT, our primary goal was to computationally redesign the S1 binding pocket of KumaWT such that it would prefer a PQ dipeptide motif over the native PR or PK substrates. Using the Rosetta Molecular Modeling Suite, we modeled the PR dipeptide in the S1 binding pocket of KumaWT using this enzyme's solved crystal structure (PDB ID: 1T1E). This revealed that two negatively-charged amino acids, D358 and D368, likely facilitate binding of the positively charged amino acids in the P1 position (FIG. 2A). The native specificity for proline at P2 appears to be derived in large part from a hydrophobic interaction of this amino acid residue with the aromatic ring of W318 in the S2 pocket of the enzyme. As specificity of the P1 position for proline is desired in our enzyme variant, we maintained this native tryptophan during the design of the S1 pocket.

To redesign the KumaWT substrate specificity of the S1 pocket to prefer glutamine at the P1 position, we generated theoretical mutations in the KumaWT binding pocket using the Foldit interface to the Rosetta Molecular Modeling Suite. A tetrapeptide that represents a common immunogenic motif found throughout α-gliadin, PQLP (SEQ ID NO: 68), was modeled into the P2 to P2′ active site positions. This structure already contained a polypeptide bound in the active site, so the residues of this polypeptide were mutated using Rosetta to the PQLP (SEQ ID NO: 68) tetrapeptide motif. A total of 75 residues within an 8 Å sphere of the tetrapeptide were randomized to any of the 20 naturally occurring amino acids in order to find mutations that would favor binding of glutamine in the S1 pocket. These mutations were accepted if the overall energy of the new enzyme-PQLP substrate complex was either reduced relative to the native substrate, or was not increased by more than 5 Rosetta energy units. To accommodate the smaller, neutral amino acid glutamine, we focused our computational efforts on 1) removing the negative charge of the S1 pocket during the design process, 2) filling in open space that resulted from the replacement of the large amino acid arginine with glutamine, and 3) providing hydrogen bonds to the amide functional group of the glutamine. This computational modeling yielded 107 novel designs containing from 1 to 7 simultaneous mutations. These designed proteins were then constructed and their catalytic activity against a PQLP (SEQ ID NO: 68) peptide was assessed.

In order to test the activity of each of these designed proteases against the PQLP (SEQ ID NO: 68) motif, the desired mutations were incorporated into the native nucleotide sequence using site directed mutagenesis, and mutant enzyme variants were produced in E. coli BL21(DE3) cells. These enzyme variants were then screened for enzymatic activity in clarified whole cell lysates at pH 4 using the fluorescently quenched α-gliadin hexapeptide analogue QXL520-PQPQLP-K(5-FAM)-NH2 (FQ) (SEQ ID NO: 69) as a substrate. Of the 107 enzyme variants tested in this assay, 13% resulted in a loss of enzymatic function, 32% did not demonstrate a significant difference in activity relative to KumaWT, and 55% resulted in an increase in observed activity against this substrate. Twenty-eight of the most promising enzyme variants that exhibited a significant increase in activity in cell lysates were then purified in order to obtain an accurate comparison of enzymatic activity to that of KumaWT. After purification and correction for protein concentration, the activities of these enzymes ranged from 2-fold to 120-fold more active than KumaWT (Table 1). The most active variant, which was named KumaMax™, was selected for further characterization.

| | Fold Change in |
|---|---|
| | Activity of PQ |
| | Hydrolysis |
| | Relative to |
| Mutations to Wild Type Kumamolysin-As | Wild Type |
| (Preprocessed) | Kumamolysin-As |
| Wild Type (WT) | 1.0 |
| T357A | 2.0 |
| G319S, D368S | 2.0 |
| D358G | 3.0 |
| D293A | 3.0 |
| D358N | 4.0 |
| G319S, S354N, D358G, D368H | 5.0 |
| D358G, D368H | 6.0 |
| G319S, D358G, D368H | 7.0 |
| N291D, Q361D | 7.5 |
| S354N, D358G, D368H | 9.0 |
| N291D | 10.0 |
| N291D, D293A, Q361D, D358N | 14.8 |
| N291D, D293A | 15.0 |
| N291D, D293A, D358G, Q361D | 15.0 |
| N291D, D358N | 18.9 |
| N291D, Q361D, D358G | 20.0 |
| N291D, G319S, D358G, Q361D, D368H | 23.1 |
| N291D, D293A, D358N | 24.0 |
| S262G, T292S, N291D, G319S, D358G, | 29.0 |
| D368H | |
| N291D, D293A, G319S, D358G, Q361D, | 40.9 |
| D368H | |
| T292S, N291D, G319S, D358G, D368H | 49.0 |
| N291D, G319S, S354N, D358G, Q361D, | 50.0 |
| D368H | |
| N291D, G319S, S354N, D358G, D368H | 54.6 |
| N291D, D293A, G319S, S354N, D358G, | 58.0 |
| Q361D, D368H | |
| D293T, N291D, G319S, D358G, D368H | 58.0 |
| S262K, D293N, N291D, G319S, D358G, | 62.0 |
| D368H | |
| N291D, G319S, D358G, D368H | 93.0 |
| V119D, S262K, D293T, N291D, G319S, | 120.0 |
| D358G, D368H | |

Table 1 Fold change in hydrolytic activity on PQ motif of all purified and sequenced mutants, relative to wild type Kumamolysin-As. These are the fold-change results (calculated as described in Supplementary Table 1) for all mutants that were purified, sequenced, and tested against wild-type Kumamolysin in the pure protein assay. The assay took place at pH 4, with enzyme final concentration of 0.0125 mg/mL and substrate concentration of 5 μM.

KumaMax™ contains seven mutations from the wild-type amino acid sequence: V119D, S262K, N291D, D293T, G319S, D358G, D368H (FIG. 2B). Of these, the mutations G319S, D358G, and D368H appear to synergistically introduce a new hydrogen bond with the desired glutamine residue at position P1. As modeled, the G319S mutation appears to introduce a hydroxyl group that is located 2.5 Å from the carbonyl oxygen of the glutamine amide, potentially contributing a new hydrogen bond that interacts with glutamine in the P1 pocket. The D368H mutation is predicted to stabilize the serine hydroxyl, and its position in the active site is in turn sterically allowed by the D358G mutation. In addition to providing a novel desired interaction with glutamine as modeled, these three mutations also remove the two acidic residues predicted to stabilize the positively charged arginine residue in the native KumaWT substrate (FIG. 2). V119D, which was unexpectedly incorporated during site directed mutagenesis, is located in the propeptide domain and therefore does not affect catalytic activity of the mature enzyme. The other three mutations do not make direct contacts with residues in the P2-P2′ pockets, and therefore likely introduce interactions with other components of the hexapeptide, the fluorophore, or the quencher. It is clear that these mutations are important for the overall catalytic enhancement observed, as the G319S/D358G/D368H triple mutant alone demonstrated only a 7-fold increase in catalytic activity over KumaWT; roughly 17-fold lower than that determined for KumaMax™

### Kinetic Characterization and Substrate Specificity

The catalytic efficiencies for KumaMax™ and KumaWT against the FQ immunogenic gluten substrate analogue, as calculated by fitting a velocity versus substrate curve over 6-100 μM substrate, were found to be 568 M⁻¹s⁻¹ and 4.9 M⁻¹s⁻¹, respectively (Table 2) These values are consistent with the observation that KumaMax™ demonstrated a 120-fold increase in enzymatic activity towards the FQ substrate in the initial activity screen mentioned above. Unfortunately, no significant saturation of velocity at these substrate concentrations was observed, and therefore the individual kinetic constants k_{cat} and K_{M} could not be determined. This is not surprising since previous analyses of the kinetic constants of KumaWT report a Km of 40 μM. Therefore, no significant saturation would be expected at substrate concentrations less than 100 μM.

| | Catalytic Efficiency M⁻¹s⁻¹ | | | | |
|---|---|---|---|---|---|
| | Qu- | Suc- | | Suc- | Suc- |
| | PQPQLP- | APQ- | Suc-APR- | APE- | AQP- |
| | Fl | pNA | pNA | pNA | pNA |
| KumaWT | 4.9 ± 0.2 | n.d. | 131.8 ± 3.8 | 4.0 ± 0.1 | n.d. |
| KumaMax ™ | 568.5 ± 14.6 | 6.7 ± 0.4 | n.d. | 1.4 ± 0.2 | n.d. |

Table 2. Kinetic Constants of peptide substrates for KumaMax™ and KumaWT. The catalytic efficiency (k_{cat}/K_{M} M⁻¹s⁻¹) for both KumaWT and KumaMa™ for the fluorescently (Fl) quenched (Qu) PQPQLP (SEQ ID NO: 34) peptide was fit to a linear curve as no saturation was observed up to 100 μM substrate. The fluorescence signal was quantified as described in Materials and Methods with a standard curve that accounted for substrate quenching of product fluorescence. The catalytic efficiency for the pNA-linked peptides was determined in a similar manner, and is described in the Materials and Methods. All fits had at least six independently measured rates with an R² greater than 0.9. n.d. not detected.

While the increased activity of KumaMax™ compared to KumaWT against the fluorescently quenched PQPQLP (SEQ ID NO: 34) hexapeptide substrate suggests that KumaMax has increased preference for a PQ dipeptide motif, it does not report directly on substrate specificity. To confirm that the specificity of KumaMax™ had indeed been altered to prefer the PQ dipeptide over the native PR dipeptide of KumaWT, four peptides in the form of Succinyl-Alanine-P2-P1-P1′ were provided as substrates to both enzymes in order to assess P2 and P1 specificity. These peptides contained the reporter p-nitroaniline (pNA) at the P1′ position, which allows for a spectrometric readout of peptide cleavage. The four peptides harbored the following amino acids at the P2 and P1 positions, respectively: proline-glutamine (PQ), proline-arginine (PR), glutamine-proline (QP), and proline-glutamate (PE). Catalytic efficiencies were calculated for each substrate and are reported in Table 2. As in the determination of catalytic activities against the FQ substrate, no saturation of activity on these peptides by KumaWT or KumaMax™ was observed. This suggests that the pNA group may partially disrupt binding in the P1′ pocket, since substrate concentrations up to 1 mM were tested, well beyond saturation levels previously reported for alternative KumaWT substrates.

In this specificity assay, KumaMax™ demonstrated its highest level of activity on the PQ substrate, the dipeptide that it had been designed to prefer. While KumaMax™ was not explicitly designed to demonstrate a decrease in specificity for the PR motif or for other motifs, its increased specificity for PQ could decrease its activity for non-targeted motifs. Indeed, KumaMax™ exhibited no significant catalytic activity against the QP or PR substrates in this assay (Table 2). Consistent with previous reports¹⁴, KumaWT exhibited its highest level of activity on the PR motif. KumaWT demonstrated significantly lower levels of activity on the three other peptide substrates. While catalytic activity of KumaWT on the PQ dipeptide motif has previously been reported¹⁴, no significant activity on the PQ dipeptide substrate was observed in this assay, which may be due to disruptive effects of pNA on the binding of this peptide to the enzyme active site. Both enzymes demonstrated activity towards the isosteric substrate PE, which is predicted to have neutral charge at pH 4; however, KumaMax™ demonstrated a roughly 5-fold decrease in activity on the PE peptide substrate compared to its activity on the PQ substrate, which illustrates its exquisite selectivity for the PQ dipeptide motif.

As discussed previously, there are several enzymes currently being explored as OET for Celiac disease. Two of these enzymes are engineered forms of the prolyl endopeptidase SC PEP and the glutamine-specific endoprotease EP-B2¹⁵. To compare the catalytic efficiencies of these proteases to that of KumaMax™, the native SC PEP and EP-B2 enzymes were expressed in E. coli BL21(DE3) cells, purified, and their catalytic activities assessed. SC-PEP demonstrated a catalytic efficiency of 1.6 M⁻¹s⁻¹ on the FQ gluten substrate analogue at pH 4, which represents a roughly 350-fold lower level of activity on this substrate than KumaMax™. At pH 4, SC PEP did not exhibit any significant activity on any of the four pNA linked peptide substrates, including QP. Although previous studies using similar pNA-linked peptides have demonstrated activity of SC PEP on these substrates, those assays were performed at a pH of 4.5 or higher¹⁵. Like other groups, we found that SC PEP demonstrated significant levels of activity on the QP substrate at pH 7, with a catalytic efficiency of 2390 M⁻¹s⁻¹, thereby confirming that this recombinant SC PEP was fully functional (data not shown). This is consistent with previous literature reporting that SC PEP has low to negligible levels of catalytic activity in the pH range of the stomach, and is thus only expected to be effective once α-gliadin peptides have reached the small intestine^{15,16}.

For EP-B2, only very low levels of activity were detected on the FQ substrate at pH 4, and no activity on any of the four pNA peptide substrates was observed (data not shown). This is inconsistent with previous reports of EP-B2 activity using comparable substrates¹¹. EP-B2 is a difficult enzyme to purify, as it forms inclusion bodies in E. coli and requires refolding to obtain active enzyme. We were unable to obtain soluble protein using previously reported methods for the refolding of EP-B2^{11,17,18}, so we used an on-column refolding process which resulted in soluble protein produced. Although this soluble EP-B2 demonstrated the expected self-processing activity at pH 4¹¹ (data not shown), the lack of activity of this enzyme suggests that it may not have refolded properly using our methods. This could be due to alternative N and C-terminal tags arising from the use of different protein expression vectors and warrants further investigation.

### Protease Stability

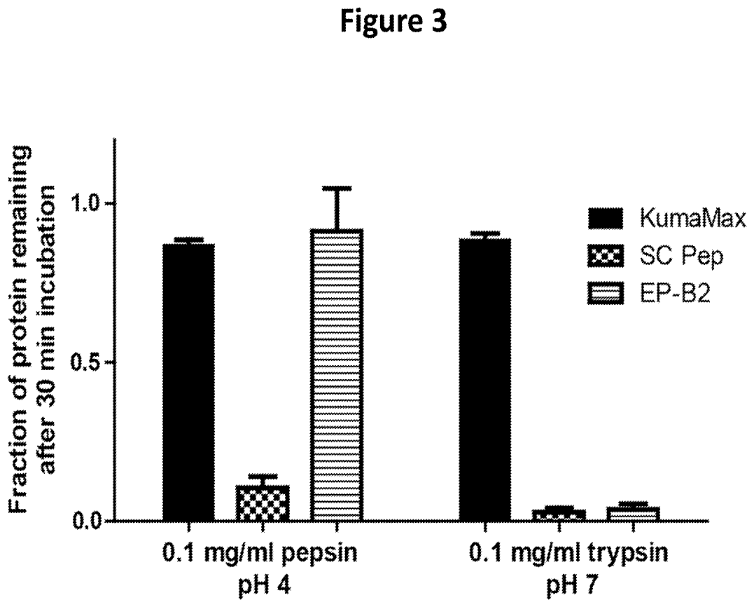

In addition to demonstrating catalytic activity at low pH, any protein therapeutic intended for use in the human digestive tract must exhibit resistance to degradation by digestive proteases. Two of the most abundant proteases in the stomach and small intestine are pepsin and trypsin, respectively. Pepsin demonstrates optimal proteolytic activity at the low pH range of the stomach, while trypsin is primarily active at the more neutral pH of the small intestine. To assess the resistance of KumaMax™ to degradation by these proteases, 0.01 or 0.1 mg/mL of KumaMax™ were incubated with each protease, in their respective optimal pH ranges, at 0.1 mg/mL, which is a physiologically relevant concentration of both pepsin and trypsin. SC PEP and EP-B2 were included as controls, as EP-B2 has been established to be resistant to pepsin but susceptible to trypsin, and SC PEP demonstrates susceptibility to both proteases^{11,15}. Each protein was incubated in the presence or absence of the respective protease for 30 minutes, after which the proteins were heat inactivated and the remaining non-proteolyzed fraction determined using an SDS-PAGE gel (FIG. 3).

In this assay, KumaMax™ demonstrated high stability against both pepsin and trypsin, with roughly 90% intact protein remaining after the half hour incubation with either protease (FIG. 3). Consistent with previous reports, SC PEP exhibited susceptibility to both pepsin and trypsin, with less than 20% of the enzyme remaining after incubation with these proteases. As expected, trypsin efficiently proteolyzed EP-B2 with less than 10% remaining after incubation, but no significant degradation of EP-B2 was observed in the presence of pepsin. To confirm that observed protein degradation was due to protease activity and not to enzymatic self-processing, each enzyme was incubated at either pH 4 or 7 and apparent proteolysis was analyzed in the absence of other proteases over the course of an hour (data not shown). KumaMax™ and EP-B2, but not SC PEP, demonstrated self-processing from the pro-peptide to the active enzyme form in fewer than 10 minutes at pH 4. All three proteins remained >90% stable over the course of the hour. None of these proteins showed significant levels of self-processing or proteolysis during incubation for one hour at pH 7.

### Degradation of an Immunogenic α9-Gliadin Peptide

The significant level of catalytic activity exhibited by KumaMax™ on immunogenic peptide analogues (Table 2) demonstrates promise for the use of KumaMax™ as a therapeutic in OET for gluten intolerance. However, these assays do not directly assess the ability of this enzyme to degrade relevant immunogenic peptides derived from gluten. Therefore, we examined the direct proteolytic activity of KumaMax™ towards an immunodominant peptide present in α9-gliadin, QLQPFPQPQLPY (SEQ ID NO: 70).

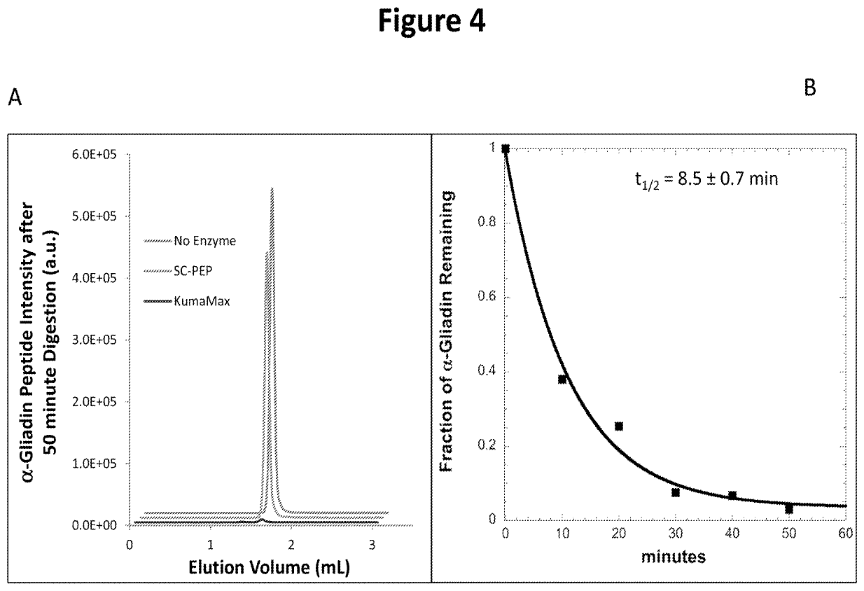

KumaMax was incubated with 500 μM of the α9-gliadin peptide at 37° C. in pH 4 at roughly a 1:100 enzyme to peptide molar ratio, which represents a physiologically relevant concentration of this peptide in the human stomach. SC PEP was included in this experiment for the sake of comparison, since this enzyme demonstrates significantly less activity against the FQ substrate than KumaMax™. Samples from the incubation were quenched every 10 minutes in 80% acetonitrile to halt the proteolysis reaction. The remaining fraction of intact immunogenic peptide was determined using high-performance liquid-chromatography mass spectroscopy, in which the M+H parent ion of the α9-gliadin peptide was monitored. KumaMax™ demonstrated a high level of activity against the immunogenic peptide in this assay, as over 95% of the immunogenic peptide had been proteolyzed after a 50 minute incubation with KumaMax™ while no significant degradation of the peptide was observed in the presence of SC PEP or in the absence of protease (FIG. 4A). The half-life of the peptide in the presence of KumaMax™ was determined by plotting the fraction of peptide remaining against the incubation time, and was calculated to be 8.5±0.7 minutes (FIG. 4B).

### Discussion

Enzyme therapy is an attractive method for the treatment of Celiac disease since this form of treatment would not require intravenous injection. However, it is a challenge to identify an appropriate protease for use in OET that demonstrates all the properties necessary to be an effective therapeutic for Celiac disease. Specifically, an ideal protease for use in OET would maintain activity in a pH range from 2-4 at 37° C. and would resist degradation by common digestive proteases. In addition, the protein therapeutic would ideally demonstrate stringent specificity for a common motif found in immunogenic gluten-derived peptides. Finally, the protein should be easily produced using recombinant methods. While it is unlikely that a single natural enzyme will encompass all of these properties, we demonstrate that a protein containing several of these important characteristics can be engineered to demonstrate the lacking qualities through computational analysis, mutagenesis, and screening.

The engineered protease, KumaMax™, demonstrated a high level of activity on, and specificity towards, the desired PQ dipeptide motif (Table 2). The specificity for the PQ motif, as opposed to the native PR motif, potentially derives from the addition of new hydrogen bonds in the S1 pocket of KumaMax that, as modeled, make direct contacts with the glutamine in this dipeptide motif (FIG. 2B). This specificity switch not only directs activity against a motif found commonly throughout gluten, but it also greatly decreases activity against non-targeted substrates (Table 2). The inability for an oral protease to recognize non-targeted substrates is an important characteristic as it reduces competitive inhibition by the large number of other peptides produced in the stomach during digestion of a meal. KumaMax™ or KumaWT can potentially act as platforms for engineering greater specificity, as KumaWT has demonstrated some level of selectivity beyond the P2 and P1 sites¹⁴. Using this method, a panel of customized proteases specific for unique immunogenic epitopes could be generated.

### Methods

### Protein Expression and Purification

The genes encoding each protein of interest, harbored in the pET29b plasmid, were transformed into Escherichia coli BL21 (DE3) cells. Individual colonies were picked, inoculated into Terrific Broth™ with 50 μg/L Kanamycin (TB+Kan), and incubated overnight at 37° C. 500 uL of the overnight culture was added to 500 mL autoinduction media (5 g tryptone, 2.5 g yeast extract, 465 mL ddH₂O), and shaken at 37° C. for roughly 4 hours, then the autoinduction components were added (500 uL MgSO₄, 500 uL 1000× trace metals, 25 mL 20×NPS, 10 mL 20×5052, 500 uL 50 mg/mL Kan). The cultures were then shaken at 18° C. for 30 hours before being spun down. Pellets were resuspended in 10 mL 1×PBS, then lysed via sonication with 5 mL lysis buffer (50 mM HEPES, 500 mM NaCl, 1 mM bME, 2 mg/mL lysozyme, 0.2 mg/mL DNase, ddH₂O) and spun down. The proteins were then purified over 1 mL TALON cobalt affinity columns. KumaMax, KumaWT, and SC Pep were washed three times with 20 mL wash buffer (10 mM imidazole, 50 mM HEPES, 500 mM NaCl, 1 mM bME, ddH₂O), and then eluted in 15 mL of elution buffer (200 mM imidazole, 50 mM HEPES, 500 mM NaCl, 1 mM bME). EP-B2 had to be refolded on the column, so after lysis the pellets were resuspended in 10 mL of EP-B2 buffer, which differs from the wash buffer only in that it is diluted in guanidine hydrochloride instead of ddH₂O to allow for denaturation of the EP-B2 inclusion bodies. This resuspension was pelleted, and the supernatant (containing denatured EP-B2) was filtered with a 0.8 m filter onto the column. EP-B2 was washed once with 20 mL of the EP-B2 buffer, before being washed twice with 20 mL of the wash buffer to refold the protein on the column. Protein was eluted with 15 ml of the elution buffer. All proteins were concentrated from 15 mL down to ~500 uL, then dialyzed once in 1 L dialysis buffer (20% glycerol, 50 mM HEPES, 500 mM NaCl, 1 mM bME). Protein concentration was calculated spectrophotometrically with extinction coefficients of 53,985 M⁻¹ cm⁻¹ for KumaWT and all KumaWT variants, 152,290 M⁻¹ cm⁻¹ for SC Pep, and 58,245 M⁻¹ cm⁻¹ for EP-B2.

### Screening Method

Kunkel mutagenesis was used to generate mutations to KumaWT. Individual colonies picked from plates were grown up in 96-deep well plates. After lysing the cells with Triton buffer (1% 100× Triton, 1 mg/mL lysozyme, 0.5 mg/mL DNase, 1×PBS), the supernatant was adjusted to pH 4 with a 100 mM sodium acetate buffer. To crudely screen for activity against the FQ substrate, 10 uL of supernatant was added to 90 uL of 5 M substrate in a 96-well black plate, and the fluorescence was measured at 30-second intervals for 1 hour.

### Purified Enzyme Assay

The variants of Kumamolisin-As that displayed the most activity on the FQ substrate in the activity screen were sequenced, then purified in small scale. 500 uL of TB+Kan overnight cultures were added to 50 mL TB+Kan and grown at 37° C. until reaching an optical density of 0.5-0.8. IPTG was added to 0.5 mM, and the cultures were expressed at 22° C. for 16-24 hours. The cells were spun down, resuspended in 500 uL of wash buffer (1×PBS, 5 mM imidazole, ddH₂O), transferred to a 2 mL Eppendorf tube, and lysed in 1 mL lysis buffer (1×PBS, 5 mM imidazole, 2× Bug Buster™, 2 mg/mL lysozyme, 0.2 mg/mL DNase, ddH₂O). After centrifugation, the supernatant was decanted into a fresh tube. Columns with 200 uL of TALON cobalt resin were placed in Eppendorf tubes, and the supernatant was poured over the columns and rocked for 20 minutes before spinning down and discarding the flow-through. The proteins were washed three times with 500 uL wash buffer, discarding the flow-through between washes. Enzymes were eluted in 200 uL elution buffer (1×PBS, 200 mM imidazole, dd H₂O), and concentrations were calculated spectrophotometrically using an extinction coefficient of 53,985 M⁻¹ cm⁻¹.

For the assay, the Kumamolisin-As mutants were incubated for 15 minutes in pH 4 100 mM sodium acetate buffer. Enzyme was added to 5 μM substrate so that the final protein concentration was 0.0125 mg/mL. The fluorescence was measured at 30-second intervals for 1 hour.

### Kinetic Characterization

Enzyme variant proclivity for gluten degradation was measured by hydrolysis of the fluorescently quenched α-gliadin hexapeptide analogue QXL520-PQPQLP-K(5-FAM)-NH2 (FQ) (SEQ ID NO: 69) as a substrate. Each enzyme was incubated at room temperature for 15 minutes in 100 mM pH 4 sodium acetate buffer. After 15 minutes, 50 uL of fluorescent substrate was added ranging in final concentration between 100, 50, 25, 12.5, 6.25, and 0 μM peptide, and maintaining concentrations of 0.05 μM KumaMax™, 0.5 μM KumaWT, 0.5 μM SC Pep, and 0.5 μM EP-B2 across all variations in substrate concentration. The plate was read immediately on the spectrophotometer for an hour, using 455 nm wavelength for excitation and reading 485 nm wavelength for emission.

The enzymes were also tested for specificity to different dipeptide motifs using a variety of chromogenic substrates that release p-nitroaniline (pNA) upon hydrolysis: [Suc-APQ-pNA], [Suc-AQP-pNA], [Suc-APE-pNA], and [Suc-APR-pNA]. Again, each enzyme was incubated at room temperature for 15 minutes in 100 mM pH 4 sodium acetate buffer. After 15 minutes, 20 μL of substrate was added to the enzyme incubation so that the final concentrations of substrate ranged between 1000, 500, 250, 125, 62.5, 31.25, 15.625, and 0 μM, and all enzymes being tested ended in a concentration of 0.5 μM. The plate was read immediately on the spectrophotometer for an hour, monitoring absorption by the reactions at 385 nm.

The standard curve for the fluorescent peptide involved mixing substrate and product together at varying concentrations in pH 4 buffer. Substrate concentrations were 100, 50, 25, 12.5, 6.25, and 0 μM, and product concentrations were 20, 5, 1.25, 0.3125, 0.078125, 0 μM.

The standard curve for the absorbent peptide involved product concentrations of 100, 50, 25, 12.5, 6.25, 3.125, 1.5625, 0.78125, 0.390625, 0.1953125, 0.09765625, and 0 μM diluted in pH 4 buffer.

### Protease Stability

Enzyme stability was determined in the presence the digestive proteases, pepsin and trypsin. KumaWT, KumaMax™, SC Pep, and EP-B2 were incubated in buffer matching the native pH environment of each digestive protease. pH 3.5 100 mM sodium acetate was used to pre-incubate the enzymes for pepsin digestion assays, and pH 7.5 dialysis buffer (see “Protein Expression and Purification”) for the trypsin digestion assays. Each experimental enzyme was incubated at 37° C. for 15 minutes in each buffer, at a concentration of 0.2 mg/mL.

After pre-incubation in the appropriate buffer, 0.1 mg/mL digestive protease was added. The reactions were done in triplicate, and were incubated at 37° C. for 30 minutes. Adding SDS and boiling for 5 minutes ensured digestive protease inactivation. An SDS-PAGE gel allowed quantification of enzyme degradation, using ImageJ.

The rate of protein self-proteolysis was determined at pH 4 and 7.5 in the absence of pepsin or trypsin. Each enzyme, at a concentration of 0.2 mg/mL, was incubated in pH 4 100 mM sodium acetate and pH 7.5 dialysis buffer. At 20, 40, and 60 minutes, timepoints were taken. SDS was added, and the aliquots were boiled for 5 minutes to ensure denaturation of the enzymes and inhibition of further self-proteolysis. Again, an SDS-PAGE gel in conjunction with ImageJ allowed quantification of enzyme self-proteolysis.

### LCMS Gliadin Degradation Assay

Enzyme activity on full-length α9-gliadin was measured using high-performance liquid-chromatography mass spectrometry. For each enzyme, 7 μL of pH 4 1M sodium acetate buffer was added to 28 μL of 5 μM enzyme, and incubated alongside separate tubes of 3 L gliadin at 37° C. for 15 minutes. Next 27 μL of each enzyme mixture, and 27 μL of dialysis buffer as a control, were added to each tube of gliadin. These were incubated once more at 37° C., and 5 μL samples were taken at 10, 20, 30, 40, and 50 minutes. Each timepoint sample was quenched in 95 μL of 80% acetonitrile with 1% formic acid and approximately 33 μM leupeptin. The samples were analyzed on the HPLC to compare gliadin degradation by the different proteases over time.

### REFERENCES

1. Armstrong, M. J., Hegade, V. S. & Robins, G. Advances in coeliac disease. Curr Opin Gastroenterol 28, 104-12 (2012).
2. Sollid, L. M. Coeliac disease: dissecting a complex inflammatory disorder. Nat Rev Immunol 2, 647-55 (2002).
3. Wieser, H. Chemistry of gluten proteins. Food Microbiol 24, 115-9 (2007).
4. Shan, L. et al. Structural basis for gluten intolerance in celiac sprue. Science 297, 2275-9 (2002).
5. Shan, L. Identification and analysis of multivalent proteolytically resistant peptides from gluten: implications for celiac sprue. Journal of Proteome Research (2005).
6. Chand, N. & Mihas, A. A. Celiac disease: current concepts in diagnosis and treatment. J Clin Gastroenterol 40, 3-14 (2006).
7. Shan, L., Marti, T., Sollid, L. M., Gray, G. M. & Khosla, C. Comparative biochemical analysis of three bacterial prolyl endopeptidases: implications for coeliac sprue. Biochem J 383, 311-8 (2004).
8. Siegel, M. et al. Rational design of combination enzyme therapy for celiac sprue. Chem Biol 13, 649-58 (2006).
9. Stepniak, D. et al. Highly efficient gluten degradation with a newly identified prolyl endoprotease: implications for celiac disease. Am J Physiol Gastrointest Liver Physiol 291, G621-9 (2006).
10. Ehren, J. et al. A food-grade enzyme preparation with modest gluten detoxification properties. PLoS One 4, e6313 (2009).
11. Bethune, M. T., Strop, P., Tang, Y., Sollid, L. M. & Khosla, C. Heterologous expression, purification, refolding, and structural-functional characterization of EP-B2, a self-activating barley cysteine endoprotease. Chem Biol 13, 637-47 (2006).
12. Okubo, A. et al. Processing, catalytic activity and crystal structures of kumamolisin-As with an engineered active site. FEBS J 273, 2563-76 (2006).
13. Gardner, J. D., Ciociola, A. A. & Robinson, M. Measurement of meal-stimulated gastric acid secretion by in vivo gastric autotitration. J Appl Physiol 92, 427-34 (2002).
14. Wlodawer, A. et al. Crystallographic and biochemical investigations of kumamolisin-As, a serine-carboxyl peptidase with collagenase activity. J Biol Chem 279, 21500-10 (2004).
15. Ehren, J., Govindarajan, S., Moron, B., Minshull, J. & Khosla, C. Protein engineering of improved prolyl endopeptidases for celiac sprue therapy. Protein Eng Des Sel 21, 699-707 (2008).
16. Bethune, M. T. & Khosla, C. Oral enzyme therapy for celiac sprue. Methods Enzymol 502, 241-71 (2012).
17. Gass, J., Vora, H., Bethune, M. T., Gray, G. M. & Khosla, C. Effect of barley endoprotease EP-B2 on gluten digestion in the intact rat. J Pharmacol Exp Ther 318, 1178-86 (2006).
18. Vora, H., McIntire, J., Kumar, P., Deshpande, M. & Khosla, C. A scaleable manufacturing process for pro-EP-B2, a cysteine protease from barley indicated for celiac sprue. Biotechnol Bioeng 98, 177-85 (2007).

We claim:

1. A polypeptide comprising an amino acid sequence at least 75% identical to an amino acid sequence according to SEQ ID NO:35, wherein
(a) the polypeptide degrades a PQPQLP (SEQ ID NO:34) peptide at pH 4;
(b) residue 278 is Ser, residue 78 is Glu, and residue 82 is Asp; and
(c) the polypeptide comprises an amino acid change from SEQ ID NO: 67 at one or more residues selected from the group consisting of 73, 102, 103, 104, 130, 165, 168, 169, 172, and 179.

2. A polypeptide comprising an amino acid sequence at least 75% identical to an amino acid sequence according to SEQ ID NO: 1, wherein
(a) the polypeptide degrades a PQPQLP (SEQ ID NO:34) peptide at pH 4;
(b) residue 467 is Ser, residue 267 is Glu, and residue 271 is Asp; and
(c) the polypeptide comprises an amino acid change from SEQ ID NO: 33 at one or more residues selected from the group consisting of 119, 262, 291, 292, 293, 319, 354, 357, 358, 361, and 368.

3. The polypeptide of claim 1 or 2, comprising an amino acid sequence at least 85% identical to an amino acid sequence according to SEQ ID NO:1 or SEQ ID NO: 35.

4. The polypeptide of claim 1 or 2, comprising an amino acid sequence at least 95% identical to an amino acid sequence according to SEQ ID NO:1 or SEQ ID NO: 35.

5. The polypeptide of claim 1, comprising an amino acid sequence according to SEQ TD NO:35.

6. The polypeptide of claim 2, comprising an amino acid sequence according to SEQ ID NO:1.

7. The polypeptide of any one of claims 1-6, wherein the polypeptide, comprises an amino acid sequence according to any one of SEQ ID NO:2-66.

8. The polypeptide of any one of claims 1-8, wherein the polypeptide comprises an amino acid sequence according to SEQ ID NO:32 or SEQ ID NO: 66.

9. A polypeptide comprising an amino acid sequence according to SEQ ID NO:1, wherein the polypeptide comprises at least one amino acid change from SEQ ID NO: 33.

10. A polypeptide comprising an amino acid sequence according to SEQ ID NO:35, wherein the polypeptide comprises at least one amino acid change from SEQ TD NO: 67.

11. The polypeptide of claim 9 or 10, comprising an amino acid sequence according to any one of SEQ ID NO:2-66.

12. The polypeptide of any one of claims 9-11, wherein the polypeptide comprises an amino acid sequence according to SEQ ID NO:32 or SEQ ID NO:66.

13. A nucleic acid encoding the polypeptide of any one of claims 1-12.

14. A nucleic acid expression vector comprising the isolated nucleic acid of claim 13.

15. A recombinant host cell comprising the nucleic acid expression vector of claim 14.

16. A pharmaceutical composition, comprising the polypeptide of any one of claims 1-12, the nucleic acid of claim 13, the nucleic acid expression vector of claim 14, and/or the recombinant host cell of claim 15, and a pharmaceutically acceptable carrier.

17. A method for treating celiac sprue, comprising administering to an individual with celiac sprue an amount effective to treat the celiac sprue of a polypeptide according to any one of claims 1-12, or a polypeptide comprising an amino acid selected from the group consisting of SEQ ID NO:33 or SEQ ID NO:67.

18. A method for treating celiac sprue, comprising administering to an individual with celiac sprue an amount effective of the pharmaceutical composition of claim 16 to treat the celiac sprue.

19. The method of any one of claims 17-18, wherein the polypeptide or the pharmaceutical composition is administered orally.

20. The method of any one of claims 17-19, wherein the polypeptide comprises an amino acid sequence according to SEQ TD NO:32 or SEQ TD NO:66.


SEQUENCE LISTING (73 sequences)

SEQ ID NO: 1 - protein, 573 aa, synthetic construct
  REGION 1..573: note = Synthetic
  VARIANT 119: note = X can be V or D
  VARIANT 262: note = X can be S, K or G
  VARIANT 291: note = X can be N or D
  VARIANT 292: note = X can be T or S
  VARIANT 293: note = X can be D, A, T, or N
  VARIANT 319: note = X can be G or S
  VARIANT 354: note = X can be S or N
  VARIANT 357: note = X can be T or A
  VARIANT 358: note = X can be D, N or G
  VARIANT 361: note = X can be Q or D
  VARIANT 368: note = X can be D, S, or H
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPXDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPXGPDGEVELDIEVAGALAPGAKFAVYFAPXXXAGFLDAITTAIHDPTLKPSVVSISWXGPEDSWTSAAIAAMNRAFLDAAALGVTVLAAAGDXGSXXGEXDGLYHVXFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 2 - protein, 573 aa, synthetic construct
  REGION 1..573: note = Synthetic
  VARIANT 119: note = X can be V or D
  VARIANT 262: note = X can be S, K or G
  SITE 291: note = MISC_FEATURE - All mutants with more than 10-fold activity have this substitution
  VARIANT 292: note = X can be T or S
  VARIANT 293: note = X can be D, A, T or N
  VARIANT 319: note = X can be G or S
  VARIANT 354: note = X can be S or N
  VARIANT 357: note = X can be T or A
  VARIANT 358: note = X can be D, N or G
  VARIANT 361: note = X can be Q or D
  VARIANT 368: note = X can be D, S or H
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPXDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPXGPDGEVELDIEVAGALAPGAKFAVYFAPDXXAGFLDAITTAIHDPTLKPSVVSISWXGPEDSWTSAAIAAMNRAFLDAAALGVTVLAAAGDXGSXXGEXDGLYHVXFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 3 - protein, 573 aa, synthetic construct; identical to SEQ ID NO: 2
  REGION 1..573: note = Synthetic
  VARIANT 119: note = X can be V or D
  VARIANT 262: note = X can be S, K, or G
  SITE 291: note = MISC_FEATURE - All mutants with more than 20-fold activity increase have this substitution together with 358 substitution
  VARIANT 292: note = X is T or S
  VARIANT 293: note = X is D, A, T or N
  VARIANT 319: note = X is G or S
  VARIANT 354: note = X is S or N
  VARIANT 357: note = X is T or A
  VARIANT 358: note = X is N or G (most have G at this position)
  VARIANT 361: note = X is Q or D
  VARIANT 368: note = X is D, S, or H

SEQ ID NO: 4 - protein, 573 aa, synthetic construct
  REGION 1..573: note = Synthetic
  VARIANT 119: note = X is V or D
  VARIANT 262: note = X is S, K or G
  SITE 291: note = MISC_FEATURE - All mutants with more than 50-fold activity increase have this substitution together with 319, 358, and 368 substitutions
  VARIANT 292: note = X is T or S
  VARIANT 293: note = X is D, A, T, or N
  VARIANT 354: note = X is S or N
  VARIANT 357: note = X is T or A
  VARIANT 361: note = X is Q or D
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPXDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPXGPDGEVELDIEVAGALAPGAKFAVYFAPDXXAGFLDAITTAIHDPTLKPSVVSISWSGPEDSWTSAAIAAMNRAFLDAAALGVTVLAAAGDXGSXGGEXDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 5 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWGGPEDSWTSAAIAAMNRAFLDAAALGVTVLAAAGDSGSTNGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 6 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTDAGFLDAITTAIHDPTLKPSVVSISWGGPEDSWTSAAIAAMNRAFLDAAALGVTVLAAAGDSGSTDGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 7 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWGGPEDSWTSAAIAAMNRAFLDAAALGVTVLAAAGDSGSTGGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 8 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWGGPEDSWTSAAIAAMNRAFLDAAALGVTVLAAAGDSGSADGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 9 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTAAGFLDAITTAIHDPTLKPSVVSISWGGPEDSWTSAAIAAMNRAFLDAAALGVTVLAAAGDSGSTDGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 10 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWSGPEDSWTSAAIAAMNRAFLDAAALGVTVLAAAGDSGSTDGEQDGLYHVSFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 11 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWGGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 12 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTDAGFLDAITTAIHDPTLKPSVVSISWGGPEDSWTSAAIAAMNRAFLDAAALGVTVLAAAGDSGSTDGEDDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 13 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTDAGFLDAITTAIHDPTLKPSVVSISWGGPEDSWTSAAIAAMNRAFLDAAALGVTVLAAAGDSGSTNGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 14 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTAAGFLDAITTAIHDPTLKPSVVSISWGGPEDSWTSAAIAAMNRAFLDAAALGVTVLAAAGDSGSTDGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 15 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTAAGFLDAITTAIHDPTLKPSVVSISWGGPEDSWTSAAIAAMNRAFLDAAALGVTVLAAAGDSGSTNGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 16 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWSGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 17 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWGGPEDSWTSAAIAAMNRAFLDAAALGVTVLAAAGDNGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 18 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTDAGFLDAITTAIHDPTLKPSVVSISWGGPEDSWTSAAIAAMNRAFLDAAALGVTVLAAAGDSGSTGGEDDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 19 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTDAGFLDAITTAIHDPTLKPSVVSISWSGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 20 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTAAGFLDAITTAIHDPTLKPSVVSISWGGPEDSWTSAAIAAMNRAFLDAAALGVTVLAAAGDSGSTNGEDDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 21 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWSGPEDSWTSAAIAAMNRAFLDAAALGVTVLAAAGDNGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 22 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTAAGFLDAITTAIHDPTLKPSVVSISWGGPEDSWTSAAIAAMNRAFLDAAALGVTVLAAAGDSGSTGGEDDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 23 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTDAGFLDAITTAIHDPTLKPSVVSISWSGPEDSWTSAAIAAMNRAFLDAAALGVTVLAAAGDSGSTGGEDDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 24 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTDAGFLDAITTAIHDPTLKPSVVSISWSGPEDSWTSAAIAAMNRAFLDAAALGVTVLAAAGDNGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 25 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTAAGFLDAITTAIHDPTLKPSVVSISWSGPEDSWTSAAIAAMNRAFLDAAALGVTVLAAAGDSGSTGGEDDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 26 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTDAGFLDAITTAIHDPTLKPSVVSISWSGPEDSWTSAAIAAMNRAFLDAAALGVTVLAAAGDNGSTGGEDDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 27 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTAAGFLDAITTAIHDPTLKPSVVSISWSGPEDSWTSAAIAAMNRAFLDAAALGVTVLAAAGDNGSTGGEDDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 28 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDSDAGFLDAITTAIHDPTLKPSVVSISWSGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 29 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTTAGFLDAITTAIHDPTLKPSVVSISWSGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 30 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPGGPDGEVELDIEVAGALAPGAKFAVYFAPDSDAGFLDAITTAIHDPTLKPSVVSISWSGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 31 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPKGPDGEVELDIEVAGALAPGAKFAVYFAPDTNAGFLDAITTAIHDPTLKPSVVSISWSGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 32 - protein, 573 aa, synthetic construct
  REGION 1..573: note = Synthetic
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPDDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPKGPDGEVELDIEVAGALAPGAKFAVYFAPDTTAGFLDAITTAIHDPTLKPSVVSISWSGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 33 - protein, 573 aa, Homo sapiens
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPVDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWGGPEDSWTSAAIAAMNRAFLDAAALGVTVLAAAGDSGSTDGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 34 - protein, 6 aa, synthetic construct
  REGION 1..6: note = Synthetic
PQPQLP

SEQ ID NO: 35 - protein, 384 aa, synthetic construct
  REGION 1..384: note = Synthetic
  VARIANT 73: note = X can be S, K or G
  VARIANT 102: note = X can be N or D
  VARIANT 103: note = X can be T or S
  VARIANT 104: note = X can be T or S
  VARIANT 130: note = X can be G or S
  VARIANT 165: note = X can be S or N
  VARIANT 168: note = X can be T or A
  VARIANT 169: note = X can be D, N or G
  VARIANT 172: note = X can be Q or D
  VARIANT 179: note = X can be D, S, or H
AAPTAYTPLDVAQAYQFPEGLDGQGQCIAIIELGGGYDEASLAQYFASLGVPAPQVVSVSVDGASNQPTGDPXGPDGEVELDIEVAGALAPGAKFAVYFAPXXXAGFLDAITTAIHDPTLKPSVVSISWXGPEDSWTSAAIAAMNRAFLDAAALGVTVLAAAGDXGSXXGEXDGLYHVXFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 36 - protein, 384 aa, synthetic construct
  REGION 1..384: note = Synthetic
  VARIANT 73: note = X can be S, K or G
  SITE 102: note = MISC_FEATURE - All mutants with more than 10-fold activity have this substitution
  VARIANT 103: note = X can be T or S
  VARIANT 104: note = X can be D, A, T or N
  VARIANT 130: note = X can be G or S
  VARIANT 165: note = X can be S or N
  VARIANT 168: note = X can be T or A
  VARIANT 169: note = X can be D, N, or G
  VARIANT 172: note = X can be Q or D
  VARIANT 179: note = X can be D, S, or H
AAPTAYTPLDVAQAYQFPEGLDGQGQCIAIIELGGGYDEASLAQYFASLGVPAPQVVSVSVDGASNQPTGDPXGPDGEVELDIEVAGALAPGAKFAVYFAPDXXAGFLDAITTAIHDPTLKPSVVSISWXGPEDSWTSAAIAAMNRAFLDAAALGVTVLAAAGDXGSXXGEXDGLYHVXFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 37 - protein, 384 aa, synthetic construct; identical to SEQ ID NO: 36
  REGION 1..384: note = Synthetic
  VARIANT 73: note = X is S, K, or G
  SITE 102: note = MISC_FEATURE - All mutants with more than 20-fold activity increase have this substitution together with 358 substitution
  VARIANT 103: note = X is T or S
  VARIANT 104: note = X is D, A, T or N
  VARIANT 130: note = X is G or S
  VARIANT 165: note = X is S or N
  VARIANT 168: note = X is T or A
  VARIANT 169: note = X is N or G (most have G at this position)
  VARIANT 172: note = X is Q or D
  VARIANT 179: note = X is D, S, or H

SEQ ID NO: 38 - protein, 384 aa, synthetic construct
  REGION 1..384: note = Synthetic
  VARIANT 73: note = X is S, K, or G
  SITE 102: note = MISC_FEATURE - All mutants with more than 50-fold activity increase have this substitution together with 319, 358, and 368 substitutions
  VARIANT 103: note = X is T or S
  VARIANT 104: note = X is D, A, T, or N
  VARIANT 165: note = X is S or N
  VARIANT 168: note = X is T or A
  VARIANT 172: note = X is Q or D
AAPTAYTPLDVAQAYQFPEGLDGQGQCIAIIELGGGYDEASLAQYFASLGVPAPQVVSVSVDGASNQPTGDPXGPDGEVELDIEVAGALAPGAKFAVYFAPDXXAGFLDAITTAIHDPTLKPSVVSISWSGPEDSWTSAAIAAMNRAFLDAAALGVTVLAAAGDXGSXGGEXDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 39 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWGGPEDSWTSAAIAAMNRAFLDAAALGVTVLAAAGDSGSTNGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 40 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTDAGFLDAITTAIHDPTLKPSVVSISWGGPEDSWTSAAIAAMNRAFLDAAALGVTVLAAAGDSGSTDGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 41 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWGGPEDSWTSAAIAAMNRAFLDAAALGVTVLAAAGDSGSTGGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 42 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWGGPEDSWTSAAIAAMNRAFLDAAALGVTVLAAAGDSGSADGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 43 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTAAGFLDAITTAIHDPTLKPSVVSISWGGPEDSWTSAAIAAMNRAFLDAAALGVTVLAAAGDSGSTDGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 44 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWSGPEDSWTSAAIAAMNRAFLDAAALGVTVLAAAGDSGSTDGEQDGLYHVSFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 45 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWGGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 46 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTDAGFLDAITTAIHDPTLKPSVVSISWGGPEDSWTSAAIAAMNRAFLDAAALGVTVLAAAGDSGSTDGEDDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 47 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTDAGFLDAITTAIHDPTLKPSVVSISWGGPEDSWTSAAIAAMNRAFLDAAALGVTVLAAAGDSGSTNGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 48 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTAAGFLDAITTAIHDPTLKPSVVSISWGGPEDSWTSAAIAAMNRAFLDAAALGVTVLAAAGDSGSTDGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 49 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTAAGFLDAITTAIHDPTLKPSVVSISWGGPEDSWTSAAIAAMNRAFLDAAALGVTVLAAAGDSGSTNGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 50 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWSGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 51 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWGGPEDSWTSAAIAAMNRAFLDAAALGVTVLAAAGDNGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 52 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTDAGFLDAITTAIHDPTLKPSVVSISWGGPEDSWTSAAIAAMNRAFLDAAALGVTVLAAAGDSGSTGGEDDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 53 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTDAGFLDAITTAIHDPTLKPSVVSISWSGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 54 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTAAGFLDAITTAIHDPTLKPSVVSISWGGPEDSWTSAAIAAMNRAFLDAAALGVTVLAAAGDSGSTNGEDDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 55 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWSGPEDSWTSAAIAAMNRAFLDAAALGVTVLAAAGDNGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 56 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTAAGFLDAITTAIHDPTLKPSVVSISWGGPEDSWTSAAIAAMNRAFLDAAALGVTVLAAAGDSGSTGGEDDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 57 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTDAGFLDAITTAIHDPTLKPSVVSISWSGPEDSWTSAAIAAMNRAFLDAAALGVTVLAAAGDSGSTGGEDDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 58 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTDAGFLDAITTAIHDPTLKPSVVSISWSGPEDSWTSAAIAAMNRAFLDAAALGVTVLAAAGDNGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 59 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTAAGFLDAITTAIHDPTLKPSVVSISWSGPEDSWTSAAIAAMNRAFLDAAALGVTVLAAAGDSGSTGGEDDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 60 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTDAGFLDAITTAIHDPTLKPSVVSISWSGPEDSWTSAAIAAMNRAFLDAAALGVTVLAAAGDNGSTGGEDDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 61 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTAAGFLDAITTAIHDPTLKPSVVSISWSGPEDSWTSAAIAAMNRAFLDAAALGVTVLAAAGDNGSTGGEDDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 62 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDSDAGFLDAITTAIHDPTLKPSVVSISWSGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 63 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPDTTAGFLDAITTAIHDPTLKPSVVSISWSGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 64 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPGGPDGEVELDIEVAGALAPGAKFAVYFAPDSDAGFLDAITTAIHDPTLKPSVVSISWSGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 65 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPKGPDGEVELDIEVAGALAPGAKFAVYFAPDTNAGFLDAITTAIHDPTLKPSVVSISWSGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 66 - protein, 384 aa, synthetic construct
  REGION 1..384: note = Synthetic
AAPTAYTPLDVAQAYQFPEGLDGQGQCIAIIELGGGYDEASLAQYFASLGVPAPQVVSVSVDGASNQPTGDPKGPDGEVELDIEVAGALAPGAKFAVYFAPDTTAGFLDAITTAIHDPTLKPSVVSISWSGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 67 - protein, 384 aa, Homo sapiens
AAPTAYTPLDVAQAYQFPEGLDGQGQCIAIIELGGGYDEASLAQYFASLGVPAPQVVSVSVDGASNQPTGDPSGPDGEVELDIEVAGALAPGAKFAVYFAPNTDAGFLDAITTAIHDPTLKPSVVSISWGGPEDSWTSAAIAAMNRAFLDAAALGVTVLAAAGDSGSTDGEQDGLYHVDFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 68 - protein, 4 aa, synthetic construct
  REGION 1..4: note = Synthetic
PQLP

SEQ ID NO: 69 - protein, 6 aa, synthetic construct
  REGION 1..6: note = Synthetic
  VARIANT 1: note = N-terminal QXL520
  VARIANT 6: note = C-terminal K(5-FAM)
PQPQLP

SEQ ID NO: 70 - protein, 12 aa, synthetic construct
  REGION 1..12: note = Synthetic
QLQPFPQPQLPY

SEQ ID NO: 71 - protein, 290 aa, Homo sapiens
MVRVPVPQLQPQNPSQQQPQEQVPLVQQQQFPGQQQPFPPQQPYPQPQPFPSQQFYLQLQPFPQPQLPYPQPQLPYPQPQLPYPQPQPFRPQQPYPQSQPQYSQPQQPISQQQQQQQQQQQQKQQQQQQQQILQQILQQQLIPCRDVVLQQHSIAYGSSQVLQQSTYGLVQQLCCQQLWQIPEQSRCQAIHNVVHAIILHQQQQQQQQQQQQPLSQVSFQQPQQQYPSGQGSFQPSQQNPQAQGSVQPQQLPQFEEIRNLALETLPAMCNVYIPPYCTIAPVGIFGTNYR

SEQ ID NO: 72 - protein, 33 aa, synthetic construct
  REGION 1..33: note = Synthetic
LQLQPFPQPQLPYPQPQLPYPQPQLPYPQPQPF

SEQ ID NO: 73 - protein, 26 aa, synthetic construct
  REGION 1..26: note = Synthetic
FLQPQQPFPPQQPQQPYPQQPQQPFP